(19) United States
(12) Patent Application Publication
(10) Pub. No.: US 2015/0133664 A1
(43) Pub. Date: May 14, 2015

(54) CHEMICAL TRANSFORMATIONS OF (-)-CODEINE TO AFFORD DERIVATIVES OF CODEINE AND MORPHINE THEREOF

(71) Applicant: BOARD OF REGENTS, THE UNIVERSITY OF TEXAS SYSTEM, Austin, TX (US)

(72) Inventors: Philip Magnus, Austin, TX (US); Bahman Ghavimi-Alagha, Austin, TX (US)

(21) Appl. No.: 14/406,134

(22) PCT Filed: Jun. 6, 2013
(86) PCT No.: PCT/US13/44491

§ 371 Date: Dec. 5, 2014

Related U.S. Application Data
(60) Provisional application No. 61/656,817, filed on Jun. 7, 2012.

(51) Int. Cl. C07D 489/02; C07D 491/18

(52) U.S. Cl. CPC C07D 489/02; C07D 491/18
USPC 546/39; 546/44

(57) ABSTRACT

The present invention relates to methods for the synthesis of morphine, codeine, intermediates, salts and derivatives thereof. In preferred embodiments, the invention relates to methods for improving the efficiency, steroselectivity, and overall yield of said codeine and morphine related derivatives and intermediates thereof. The present invention relates to new codeine and morphine related derivative compositions.

### CROSS-REFERENCE TO RELATED APPLICATIONS

The present application claims the benefit of U.S. Provisional Patent Application No. 61/656,817, filed on Jun. 7, 2012, which is incorporated herein by reference [1].

### FIELD OF THE INVENTION

The present invention relates to methods and compositions for the synthesis of morphine and precursors, intermediates (including but not limited to codeine), salts, and derivatives thereof. In addition, pharmaceutical formulations comprising such compositions, as well as methods of treatment comprising administering said compositions), are contemplated. In preferred embodiments, the invention relates to methods for improving the efficiency and overall yield of said morphine, morphine related derivatives and intermediates thereof, as well as the resulting compositions for pharmaceutical formulations and human treatment (e.g. to relieve or prevent pain, to suppress coughing, etc.). The present invention relates to methods for the synthesis of morphine, codeine, intermediates, salts and derivatives thereof. In preferred embodiments, the invention relates to methods for improving the efficiency, steroselectivity, and overall yield of said codeine and morphine related derivatives and intermediates thereof. The present invention relates to new codeine and morphine related derivative compositions.

### BACKGROUND OF THE INVENTION

Morphine is one of the most important analgesics worldwide. The majority of the world's morphine supply is derived from poppy plants found in some of the more politically turbulent areas of western Asia. Codeine or 3-methylmorphine is a natural isomer of methylated morphine. While morphine remains in high demand worldwide, the lack of effective synthetic methods coupled with the aforementioned instability in areas largely responsible for the natural production of morphine illustrates the tenuous state of current means for obtaining the compound. Thus, there is a need to develop improved methods for synthesizing morphine and related derivatives for use in pharmaceutical compositions and other medical applications.

### SUMMARY OF THE INVENTION

The present invention relates to methods and compositions for the synthesis of morphine and precursors, intermediates (including but not limited to codeine), salts, and derivatives thereof. In addition, pharmaceutical formulations comprising such compositions, as well as methods of treatment comprising administering said compositions), are contemplated. In preferred embodiments, the invention relates to methods for improving the efficiency and overall yield of said morphine, morphine related derivatives and intermediates thereof, as well as the resulting compositions for pharmaceutical formulations and human treatment (e.g. to relieve or prevent pain, to suppress coughing, etc.). The present invention relates to methods for the synthesis of morphine, codeine, intermediates, salts and derivatives thereof. In preferred embodiments, the invention relates to methods for improving the efficiency, steroselectivity, and overall yield of said codeine and morphine related derivatives and intermediates thereof. The present invention relates to new codeine and morphine related derivative compositions.

In one embodiment, the invention relates to a method of preparing a carbamate derivative, comprising: a) providing (−)-codeine phosphate; and b) treating said codeine phosphate derivative under conditions so as to create a carbamate derivative. In one embodiment, said step b) comprises treating said (−)-codeine phosphate with ClCO₂Et, K₂CO₃, and chloroform in reflux. In one embodiment, said (−)-codeine phosphate has the structure:

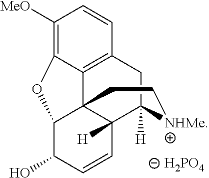

In one embodiment, said stereospecific enantiomer carbamate derivative has the structure:

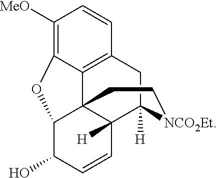

In one embodiment, the method further comprises c) treating said carbamate derivative with reducing agent, so as to create a 6,7-alkene derivative. In one embodiment, said 6,7-alkene derivative has the structure:

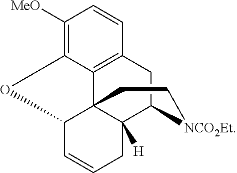

In one embodiment, the method further comprises d) treating said 6,7-alkene derivative in dioxane and water at −10° C. and 1,3-dibromo-5,5-dimethylhydantoin, so as to create a bromohydrin. In one embodiment, said bromohydrin has the structure:

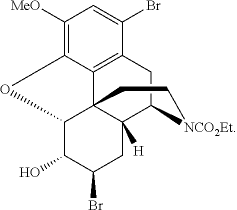

In one embodiment, the method further comprises e) treating said halohydrin with KOH, so as to create a 6,7-epoxide derivative. In one embodiment, said 6,7-epoxide derivative has the structure:

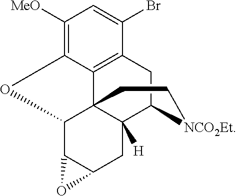

In one embodiment, the method further comprises f) treating said 6,7-epoxide derivative under such conditions to create a ring opened derivative with the structure:

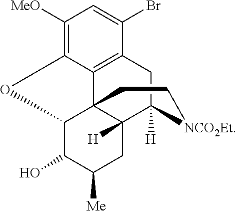

In one embodiment, said conditions comprise in dichloromethane and water with Me₃Al. In one embodiment, the method further comprises g) treating said ring opened derivative under reducing conditions, so as to create a tertiary amine derivative with the structure:

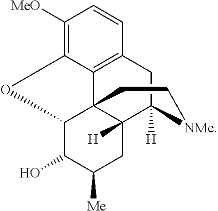

In one embodiment, said conditions comprise LiAlH₄ in THF at 0° C.

In one embodiment, the method further comprises f) treating said 6,7-epoxide derivative under such conditions to create a ring opened derivative with the structure:

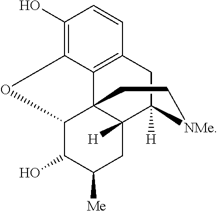

In one embodiment, said conditions comprise BBr₃ in CH₂Cl₂ under temperatures between 0 to 25° C. In one embodiment, the method further comprises f) treating said 6,7-epoxide derivative in anhydrous MeOH wth p-toluenesulfonic acid added and refluxed to create a ring opened derivative with the structure:

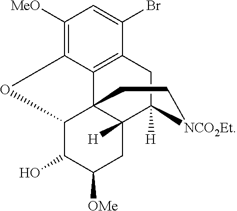

In one embodiment, the method further comprises g) treating said ring opened derivative under such reducing conditions to create a ring opened derivative with the structure:

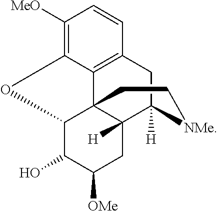

In one embodiment, said reducing conditions comprise lithium aluminum hydride in THF at room temperature under argon. In one embodiment, the method further comprises f) treating said 6,7-epoxide derivative in water and THF with methanesulfonic acid added and refluxed to create a ring opened derivative with the structure:

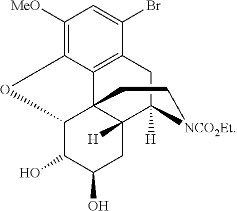

In one embodiment, the method further comprises g) treating said ring opened derivative under such reducing conditions to create a ring opened derivative with the structure:

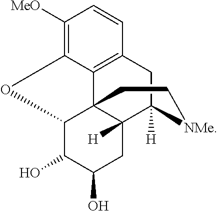

In one embodiment, said reducing conditions comprise lithium aluminum hydride in THF at room temperature under argon. In one embodiment, the method further comprises f) treating said 6,7-epoxide derivative in dichloromethane with HF pyridine added and the mixture stirred at room temperature under argon to create a ring opened derivative with the structure:

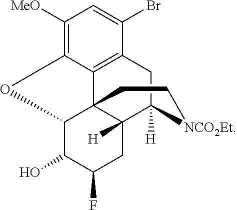

In one embodiment, the method further comprises g) treating said ring opened derivative under such reducing conditions to create a ring opened derivative with the structure:

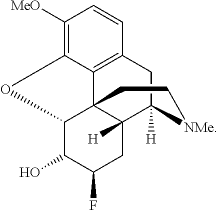

In one embodiment, the method further comprises f) treating said 6,7-epoxide derivative in THF with imidazole and KH added with subsequent addition of methyl iodide and the mixture stirred at room temperature under argon to create a ring opened derivative with the structure:

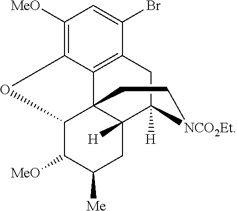

In one embodiment, the method further comprises g) treating said ring opened derivative under such reducing conditions to create a ring opened derivative with the structure:

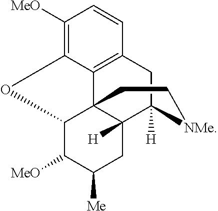

In one embodiment, said reducing conditions comprise lithium aluminum hydride in THF at room temperature under argon.

In one embodiment, the invention relates to an absolute stereoisomer composition of the formula:

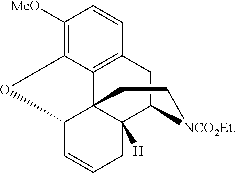

In one embodiment, the invention relates to an absolute stereoisomer composition of the formula:

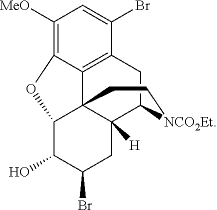

In one embodiment, the invention relates to an absolute stereoisomer composition of the formula:

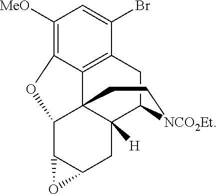

In one embodiment, the invention relates to an absolute stereoisomer composition of the formula:

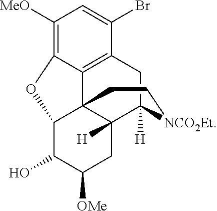

In one embodiment, the invention relates to an absolute stereoisomer composition of the formula:

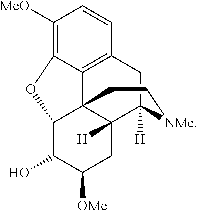

In one embodiment, the invention relates to an absolute stereoisomer composition of the formula:

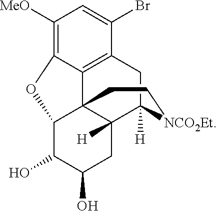

In one embodiment, the invention relates to an absolute stereoisomer composition of the formula:

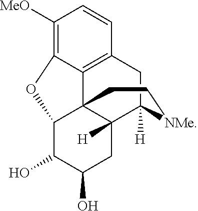

In one embodiment, the invention relates to an absolute stereoisomer composition of the formula:

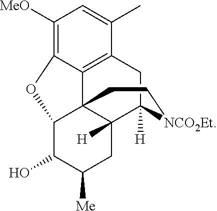

In one embodiment, the invention relates to an absolute stereoisomer composition of the formula:

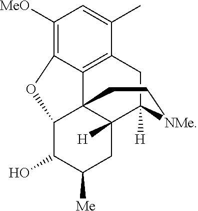

In one embodiment, the invention relates to an absolute stereoisomer composition of the formula:

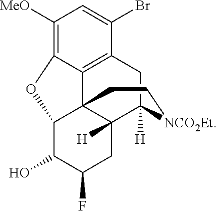

In one embodiment, the invention relates to an absolute stereoisomer composition of the formula:

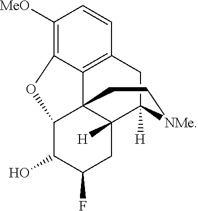

In one embodiment, the invention relates to an absolute stereoisomer composition of the formula:

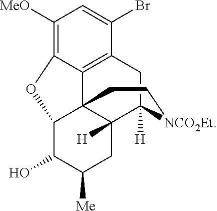

In one embodiment, the invention relates to an absolute stereoisomer composition of the formula:

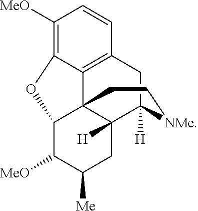

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Other non-carbon groups contemplated by the present invention as candidates for substituting into the compounds described herein include, but are not limited to oxy, amino, amido, imino, thio, thiol, sulfonyl, ammonium, sulfonium, silyl and the substituted versions of these groups.

Starting with (−)-codeine phosphate 1, it was converted into the known carbamate 3 following literature procedures (FIG. 2B) [2]. Treatment of 3 carbamate with DEAD/PPh₃/NMM/NBSH [3] gave the 6,7-alkene 4 as a single enantiomer (see FIG. 2B). This compound was previously made by total synthesis as a racemate [4]. Access to compound 4 through synthesis from codeine is much shorter, and supplies material that can be converted into derivatives for biological assays that are single optical isomers.

In some embodiments the terms alkyl, aryl, alkanediyl, alkynyl, arenediyl, aralkyl, heteroarenediyl, heteroaralkyl, heteroaryl, alkenyl, alkenediyl, alkynediyl, acyl, alkylidene, or a substituted version of any of these groups, refer to groups with a number of carbons≦20. In some embodiments the terms alkyl, aryl, alkanediyl, alkynyl, arenediyl, aralkyl, heteroarenediyl, heteroaralkyl, heteroaryl, alkenyl, alkenediyl, alkynediyl, acyl, alkylidene, or a substituted version of any of these groups, refer to groups with a number of carbons≦12. In some embodiments the terms alkyl, aryl, alkanediyl, alkynyl, arenediyl, aralkyl, heteroarenediyl, heteroaralkyl, heteroaryl, alkenyl, alkenediyl, alkynediyl, acyl, alkylidene, or a substituted version of any of these groups refer to groups with a number of carbons≦10. In some embodiments the terms alkyl, aryl, alkanediyl, alkynyl, arenediyl, aralkyl, heteroarenediyl, heteroaralkyl, heteroaryl, alkenyl, alkenediyl, alkynediyl, acyl, alkylidene, or a substituted version of any of these groups, refer to groups with a number of carbons≦8. In some embodiments, the present invention contemplates allyl, propargyl, and cyclopropyl carbinol derivatives.

In one embodiment, the invention relates to a composition of the formula (or salt thereof):

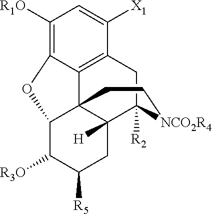

wherein R₁ is an alkyl, alkanediyl, alkynyl, aryl, arenediyl, aralkyl, heteroaryl, heteroarenediyl, heteroaralkyl, or a substituted version of any of these groups, or H; R₂ is an alkyl, alkanediyl, alkynyl, aryl, arenediyl, aralkyl, heteroaryl, heteroarenediyl, heteroaralkyl, or a substituted version of any of these groups, or H; R₃ is an alkyl, alkanediyl, alkynyl, aryl, arenediyl, aralkyl, heteroaryl, heteroarenediyl, heteroaralkyl, or a substituted version of any of these groups, or H; R₄ is an alkyl, alkanediyl, alkynyl, aryl, arenediyl, aralkyl, heteroaryl, heteroarenediyl, heteroaralkyl, or a substituted version of any of these groups; R₅ is F, Cl, Br, alkyl, alkanediyl, alkynyl, aryl, arenediyl, aralkyl, heteroaryl, heteroarenediyl, heteroaralkyl, or a substituted version of any of these groups; and X¹ is alkyl F, Cl, Br, I or equivalent leaving group. In still further embodiments, the invention relates to a composition of the formula (or salt thereof):

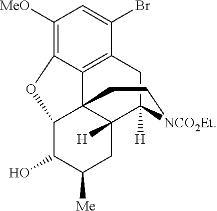

In still further embodiments, the invention relates to a composition of the formula (or salt thereof):

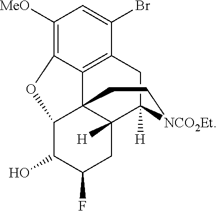

In still further embodiments, the invention relates to a composition of the formula (or salt thereof):

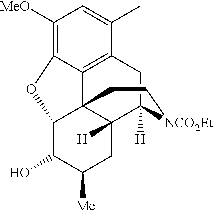

In one embodiment, the invention relates to a composition of the formula (or salt thereof):

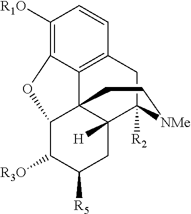

wherein R₁ is an alkyl, alkanediyl, alkynyl, aryl, arenediyl, aralkyl, heteroaryl, heteroarenediyl, heteroaralkyl, or a substituted version of any of these groups, or H; R₂ is an alkyl, alkanediyl, alkynyl, aryl, arenediyl, aralkyl, heteroaryl, heteroarenediyl, heteroaralkyl, or a substituted version of any of these groups, or H; R₃ is an alkyl, alkanediyl, alkynyl, aryl, arenediyl, aralkyl, heteroaryl, heteroarenediyl, heteroaralkyl, or a substituted version of any of these groups, or H; and R₅ is F, Cl, Br, alkyl, alkanediyl, alkynyl, aryl, arenediyl, aralkyl, heteroaryl, heteroarenediyl, heteroaralkyl, or a substituted version of any of these groups. In still further embodiments, the invention relates to a composition of the formula (or salt thereof):

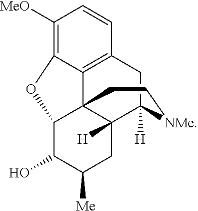

In still further embodiments, the invention relates to a composition of the formula (or salt thereof):

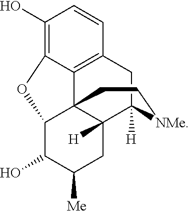

In still further embodiments, the invention relates to a composition of the formula (or salt thereof):

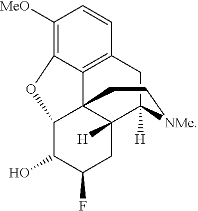

In one embodiment, the invention relates to a composition of the formula (or salt thereof):

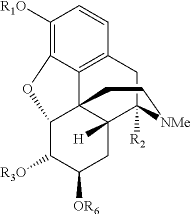

wherein R₁ is an alkyl, alkanediyl, alkynyl, aryl, arenediyl, aralkyl, heteroaryl, heteroarenediyl, heteroaralkyl, or a substituted version of any of these groups, or H; R₂ is an alkyl, alkanediyl, alkynyl, aryl, arenediyl, aralkyl, heteroaryl, heteroarenediyl, heteroaralkyl, or a substituted version of any of these groups, or H; R₃ is an alkyl, alkanediyl, alkynyl, aryl, arenediyl, aralkyl, heteroaryl, heteroarenediyl, heteroaralkyl, or a substituted version of any of these groups, or H; and R₆ is an alkyl, alkanediyl, alkynyl, aryl, arenediyl, aralkyl, heteroaryl, heteroarenediyl, heteroaralkyl, or a substituted version of any of these groups, or H. In still further embodiments, the invention relates to a composition of the formula (or salt thereof):

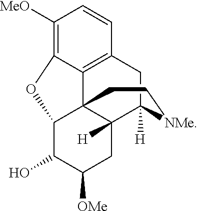

In still further embodiments, the invention relates to a composition of the formula (or salt thereof):

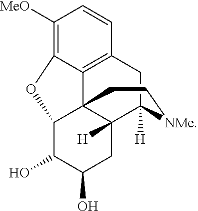

In still further embodiments, the invention relates to a composition of the formula (or salt thereof):

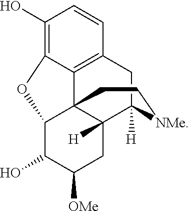

In still further embodiments, the invention relates to a composition of the formula (or salt thereof):

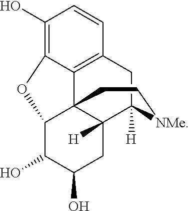

In one embodiment, the invention relates to a composition of the formula (or salt thereof):

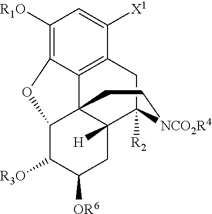

wherein R¹ is an alkyl, alkanediyl, alkynyl, aryl, arenediyl, aralkyl, heteroaryl, heteroarenediyl, heteroaralkyl, or a substituted version of any of these groups, or H; R² is an alkyl, alkanediyl, alkynyl, aryl, arenediyl, aralkyl, heteroaryl, heteroarenediyl, heteroaralkyl, or a substituted version of any of these groups, or H; R³ is an alkyl, alkanediyl, alkynyl, aryl, arenediyl, aralkyl, heteroaryl, heteroarenediyl, heteroaralkyl, or a substituted version of any of these groups, or H; R⁶ is an alkyl, alkanediyl, alkynyl, aryl, arenediyl, aralkyl, heteroaryl, heteroarenediyl, heteroaralkyl, or a substituted version of any of these groups, or H; and X¹ is alkyl F, Cl, Br, I or equivalent leaving group. In still further embodiments, the invention relates to a composition of the formula (or salt thereof):

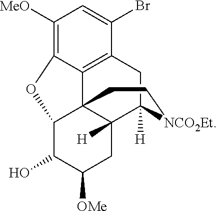

In still further embodiments, the invention relates to a composition of the formula (or salt thereof):

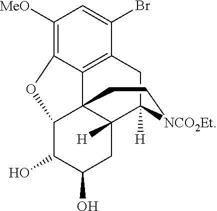

In one embodiment, the invention contemplates a method of preparing a carbamate derivative, comprising: a) providing a codeine phosphate derivative; b) treating said codeine phosphate derivative under conditions (e.g. ClCO₂Et/K₂CO₃/CHCl₃ reflux) so as to create a carbamate derivative. Some generic embodiments are shown in FIG. 2A. Some specific non-limiting examples of contemplated derivatives are shown in FIG. 2B. In one embodiment, step b) comprises treating said codeine phosphate derivative with a substituted carbonochloridate ClCO₂R⁴, where R⁴ is an alkyl, alkanediyl, alkynyl, aryl, arenediyl, aralkyl, heteroaryl, heteroarenediyl, heteroaralkyl, or a substituted version of any of these groups, potassium carbonate, and chloroform in reflux. In one embodiment, said codeine phosphate derivative has the structure:

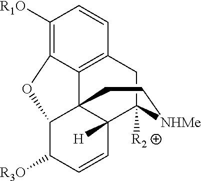

wherein R₁ is an alkyl, alkanediyl, alkynyl, aryl, arenediyl, aralkyl, heteroaryl, heteroarenediyl, heteroaralkyl, or a substituted version of any of these groups or a protecting group, or H; R₂ is alkyl, alkanediyl, alkynyl, aryl, arenediyl, aralkyl, heteroaryl, heteroarenediyl, heteroaralkyl, or a substituted version of any of these groups, or H; and R₃ is an alkyl, alkanediyl, alkynyl, aryl, arenediyl, aralkyl, heteroaryl, heteroarenediyl, heteroaralkyl, or a substituted version of any of these groups or a protecting group, or H. In one embodiment, said carbamate derivative has the structure:

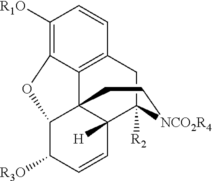

wherein R₁ is an alkyl, alkanediyl, alkynyl, aryl, arenediyl, aralkyl, heteroaryl, heteroarenediyl, heteroaralkyl, or a substituted version of any of these groups or a protecting group, or H; R₂ is alkyl, alkanediyl, alkynyl, aryl, arenediyl, aralkyl, heteroaryl, heteroarenediyl, heteroaralkyl, or a substituted version of any of these groups, or H; R₃ is an alkyl, alkanediyl, alkynyl, aryl, arenediyl, aralkyl, heteroaryl, heteroarenediyl, heteroaralkyl, or a substituted version of any of these groups or a protecting group, or H; and R₄ is an alkyl, alkanediyl, alkynyl, aryl, arenediyl, aralkyl, heteroaryl, heteroarenediyl, heteroaralkyl, or a substituted version of any of these groups. Some generic embodiments are shown in FIG. 2A. Some specific non-limiting examples of contemplated derivatives are shown in FIG. 2B. In one embodiment, the invention further comprises step c) treating said carbamate derivative with reducing agent (e.g. Treatment with DEAD/PPh₃/NMM/NBSH), so as to create a 6,7-alkene derivative. In one embodiment, said 6,7-alkene derivative has the structure:

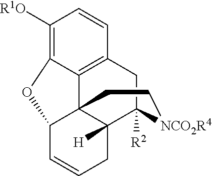

wherein R₁ is an alkyl, alkanediyl, alkynyl, aryl, arenediyl, aralkyl, heteroaryl, heteroarenediyl, heteroaralkyl, or a substituted version of any of these groups or a protecting group, or H; R₂ is alkyl, alkanediyl, alkynyl, aryl, arenediyl, aralkyl, heteroaryl, heteroarenediyl, heteroaralkyl, or a substituted version of any of these groups, or H; and R₄ is an alkyl, alkanediyl, alkynyl, aryl, arenediyl, aralkyl, heteroaryl, heteroarenediyl, heteroaralkyl, or a substituted version of any of these groups. In one embodiment, the invention further comprises step d) treating said 6,7-alkene derivative with halohydantoin (e.g. 1,3-dibromo-5,5-dimethylhydantoin), so as to create a halohydrin. Some generic embodiments are shown in FIG. 2A. Some specific non-limiting examples of contemplated derivatives are shown in FIG. 2B. In one embodiment, said halohydantoin is 1,3 dibromo-5,5 dimethylhydantoin and said halohydrin is a bromohydrin. In one embodiment, said halohydrin has the structure:

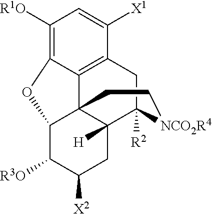

wherein R₁ is an alkyl, alkanediyl, alkynyl, aryl, arenediyl, aralkyl, heteroaryl, heteroarenediyl, heteroaralkyl, or a substituted version of any of these groups or a protecting group, or H; R₂ is alkyl, alkanediyl, alkynyl, aryl, arenediyl, aralkyl, heteroaryl, heteroarenediyl, heteroaralkyl, or a substituted version of any of these groups, or H; R₃ is an alkyl, alkanediyl, alkynyl, aryl, arenediyl, aralkyl, heteroaryl, heteroarenediyl, heteroaralkyl, or a substituted version of any of these groups or a protecting group, or H; R₄ is an alkyl, alkanediyl, alkynyl, aryl, arenediyl, aralkyl, heteroaryl, heteroarenediyl, heteroaralkyl, or a substituted version of any of these groups; X¹ is alkyl, F, Cl, Br, I or equivalent leaving group; and X² is F, Cl, Br, I or equivalent leaving group. Some generic embodiments are shown in FIG. 2A. Some specific non-limiting examples of contemplated derivatives are shown in FIG. 2B. In one embodiment, the invention further comprises step e) treating said halohydrin with strong base (e.g. KOH), so as to create a 6,7-epoxide derivative. In one embodiment, said 6,7-epoxide derivative has the structure:

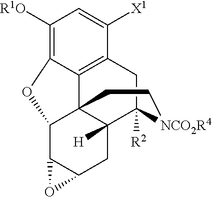

wherein R₁ is an alkyl, alkanediyl, alkynyl, aryl, arenediyl, aralkyl, heteroaryl, heteroarenediyl, heteroaralkyl, or a substituted version of any of these groups or a protecting group, or H; R₂ is alkyl, alkanediyl, alkynyl, aryl, arenediyl, aralkyl, heteroaryl, heteroarenediyl, heteroaralkyl, or a substituted version of any of these groups, or H; R₄ is an alkyl, alkanediyl, alkynyl, aryl, arenediyl, aralkyl, heteroaryl, heteroarenediyl, heteroaralkyl, or a substituted version of any of these groups. and X¹ is alkyl, F, Cl, Br, I or equivalent leaving group. Some generic embodiments are shown in FIG. 3A. Some specific non-limiting examples of contemplated derivatives are shown in FIG. 3B. In one embodiment, the invention further comprises step f) treating said 6,7-epoxide derivative under such conditions (e.g. Me₃Al/CH₂Cl₂/H₂O) to create a ring opened derivative with the structure:

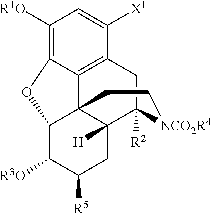

wherein R¹ is an alkyl, alkanediyl, alkynyl, aryl, arenediyl, aralkyl, heteroaryl, heteroarenediyl, heteroaralkyl, or a substituted version of any of these groups, or H; R² is an alkyl, alkanediyl, alkynyl, aryl, arenediyl, aralkyl, heteroaryl, heteroarenediyl, heteroaralkyl, or a substituted version of any of these groups, or H; R³ is an alkyl, alkanediyl, alkynyl, aryl, arenediyl, aralkyl, heteroaryl, heteroarenediyl, heteroaralkyl, or a substituted version of any of these groups, or H; R⁴ is an alkyl, alkanediyl, alkynyl, aryl, arenediyl, aralkyl, heteroaryl, heteroarenediyl, heteroaralkyl, or a substituted version of any of these groups; R₅ is F, alkyl, alkanediyl, alkynyl, aryl, arenediyl, aralkyl, heteroaryl, heteroarenediyl, heteroaralkyl, or a substituted version of any of these groups; and X¹ is alkyl, F, Cl, Br, I or equivalent leaving group. Some generic embodiments are shown in FIG. 3A. Some specific non-limiting examples of contemplated derivatives are shown in FIG. 3B. In one embodiment, the invention further comprises step g) treating said ring opened derivative under reducing conditions (e.g. LiAlH₄/THF at 0° C.), so as to create a tertiary amine derivative with the structure:

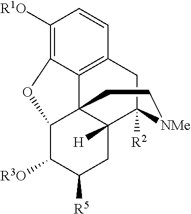

wherein R¹ is an alkyl, alkanediyl, alkynyl, aryl, arenediyl, aralkyl, heteroaryl, heteroarenediyl, heteroaralkyl, or a substituted version of any of these groups, or H; R² is an alkyl, alkanediyl, alkynyl, aryl, arenediyl, aralkyl, heteroaryl, heteroarenediyl, heteroaralkyl, or a substituted version of any of these groups, or H; R³ is an alkyl, alkanediyl, alkynyl, aryl, arenediyl, aralkyl, heteroaryl, heteroarenediyl, heteroaralkyl, or a substituted version of any of these groups, or H; and R₅ is F, alkyl, alkanediyl, alkynyl, aryl, arenediyl, aralkyl, heteroaryl, heteroarenediyl, heteroaralkyl, or a substituted version of any of these groups. Some generic embodiments are shown in FIG. 3A. Some specific non-limiting examples of contemplated derivatives are shown in FIG. 3B. In one embodiment, the invention further comprises step f) treating said 6,7-epoxide derivative under such conditions (e.g. BBr₃/CH₂Cl₂, 0 to 25° C.) to create a ring opened derivative with the structure:

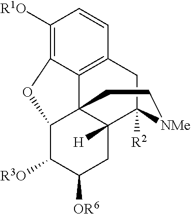

wherein R¹ is an alkyl, alkanediyl, alkynyl, aryl, arenediyl, aralkyl, heteroaryl, heteroarenediyl, heteroaralkyl, or a substituted version of any of these groups, or H; R² is an alkyl, alkanediyl, alkynyl, aryl, arenediyl, aralkyl, heteroaryl, heteroarenediyl, heteroaralkyl, or a substituted version of any of these groups, or H; R³ is an alkyl, alkanediyl, alkynyl, aryl, arenediyl, aralkyl, heteroaryl, heteroarenediyl, heteroaralkyl, or a substituted version of any of these groups, or H; and R⁶ is an alkyl, alkanediyl, alkynyl, aryl, arenediyl, aralkyl, heteroaryl, heteroarenediyl, heteroaralkyl, or a substituted version of any of these groups, or H. A generic embodiment is shown in FIG. 5. Some specific non-limiting examples of contemplated derivatives are shown in FIG. 5.

In this regard, the above-described steps can be modified to create these derivatives. Moreover, the present invention contemplates treating and/or preventing disease with morphine and codeine (and derivatives thereof) synthesized according to the above scheme and formulated as pharmaceutical formulations.

### BRIEF DESCRIPTION OF THE DRAWINGS

For a more complete understanding of the features and advantages of the present invention, reference is now made to the detailed description of the invention along with the accompanying figures.

FIG. 1 shows the atomic numbering scheme for morphine and codeine.

FIGS. 2 A and B show embodiments of the present invention for synthesizing compounds useful in the synthesis of derivatives of both morphine and codeine. FIG. 2A provides the general overall scheme, while FIG. 2B provides specific (non-limiting) examples.

FIGS. 3 A and B show embodiments of the present invention for synthesizing compounds useful in the synthesis of derivatives of both morphine and codeine. FIG. 3A provides the general overall scheme, while FIG. 3B provides specific (non-limiting) examples.

FIG. 4 provides a several specific (non-limiting) examples of additional morphine and codeine derivatives, compounds 10, 10′, 11, 11′, 12, 12′, 13, 13′, 14 and 14′.

FIG. 5 provides a general and several specific (non-limiting) examples of additional morphine and codeine derivatives that can be made from the 6,7 epoxide derivative.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “cross-conjugated” refers to a compound where in there are (at least) two double bonds that are conjugated to a “central” double bond in such a way that the π electronic system forms a bifurcation.

As used herein, “morphine” refers to a compound represented by the following chemical structure:

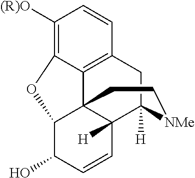

where R is H. It is not intended that the invention be limited to any particular derivative or analog of morphine or salt thereof, however every description of stereochemistry is to be understood as absolute. It is important to note that such absolute stereochemical isomer is distinct from a racemic misture of isomers. Examples of derivatives of morphine include but are in no way limited to morphine, morphine acetate, morphine citrate, morphine bitartrate, morphine stearate, morphine phthalate, morphine hydrobromide, morphine hydrobromide.2H₂O, morphine hydrochloride, morphine hydrochloride.3H₂O, morphine hydriodide.2H₂O, morphine lactate, morphine monohydrate, morphine meconate.5H₂O, morphine mucate, morphine nitrate, morphine phosphate.0.5H₂O, morphine phosphate.7H₂O, morphine salicylate, morphine phenylpropionate, morphine methyliodide, morphine isobutyrate, morphine hypophosphite, morphine sulfate.5H₂O, morphine tannate, morphine tartrate.3H₂O, morphine valerate, morphine methylbromide, morphine methylsulfonate, morphine-N-oxide, morphine-N-oxide quinate, dihydromorphine and pseudomorphine. It is not intended that the present invention be limited by the type of chemical substituent or substituents that is or are coordinated to morphine. Examples of chemical substituents include but are in no way limited to hydrogen, methyl, ethyl, formyl, acetyl, phenyl, chloride, bromide, hydroxyl, methoxyl, ethoxyl, methylthiol, ethylthiol, propionyl, carboxyl, methoxy carbonyl, ethoxycarbonyl, methylthiocarbonyl, ethylthiocarbonyl, butylthiocarbonyl, dimethylcarbamyl, diethylcarbamyl, N-piperidinylcarbonyl, N-methyl-N′-piperazinylcarbonyl, 2-(dimethylamino)ethylcarboxy, N-morpholinylcarbonyl, 2-(dimethylamino)ethylcarbamyl, 1-piperidinylcarbonyl, methylsulfonyl, ethylsulfonyl, phenylsulfonyl, 2-piperidinylethyl, 2-morpholinylethyl, 2-(dimethylamino)ethyl, 2-(diethylamino)ethyl, butylthiol, dimethylamino, diethylamino, piperidinyl, pyrrolidinyl, imidazolyl, pyrazolyl, N-methylpiperazinyl and 2-(dimethylamino)ethylamino.

As used herein, “codeine” refers to a compound represented by the following chemical structure:

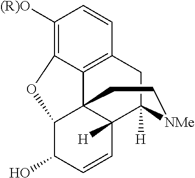

where R is CH₃, also referred to as a methyl (Me) substituent. It is not intended that the invention be limited to any particular derivative, analog of codeine or salt thereof. Examples of derivatives of codeine include but are in no way limited to codeine, codeine acetate, codeine citrate, codeine bitartrate, codeine stearate, codeine phthalate, codeine hydrobromide, codeine hydrobromide.2H₂O, codeine hydrochloride, codeine hydrochloride.3H₂O, codeine hydriodide.2H₂O, codeine lactate, codeine monohydrate, codeine meconate.5H₂O, codeine mucate, codeine nitrate, codeine phosphate.0.5H₂O, codeine phosphate.7H₂O, codeine salicylate, codeine phenylpropionate, codeine methyliodide, codeine isobutyrate, codeine hypophosphite, codeine sulfate.5H₂O, codeine tannate, codeine tartrate.3H₂O, codeine valerate, codeine methylbromide, codeine methylsulfonate, codeine-N-oxide, codeine-N-oxide quinate and pseudocodeine. It is not intended that the present invention be limited by the type of chemical substituent or substituents that is or are coordinated to codeine. Examples of chemical substituents include but are in no way limited to hydrogen, methyl, ethyl, formyl, acetyl, phenyl, chloride, bromide, hydroxyl, methoxyl, ethoxyl, methylthiol, ethylthiol, propionyl, carboxyl, methoxy carbonyl, ethoxycarbonyl, methylthiocarbonyl, ethylthiocarbonyl, butylthiocarbonyl, dimethylcarbamyl, diethylcarbamyl, N-piperidinylcarbonyl, N-methyl-N-piperazinylcarbonyl, 2-(dimethylamino)ethylcarboxy, N-morpholinylcarbonyl, 2-(dimethylamino)ethylcarbamyl, 1-piperidinylcarbonyl, methylsulfonyl, ethylsulfonyl, phenylsulfonyl, 2-piperidinylethyl, 2-morpholinylethyl, 2-(dimethylamino)ethyl, 2-(diethylamino)ethyl, butylthiol, dimethylamino, diethylamino, piperidinyl, pyrrolidinyl, imidazolyl, pyrazolyl, N-methylpiperazinyl and 2-(dimethylamino)ethylamino.

As used herein, “alkaloid” refers to a member of the class of naturally occurring chemical compounds containing basic nitrogen atoms. Alkaloids are produced by a large variety of organisms, with many exhibiting pharmacological effects. While not limiting the scope of the present invention, alkaloids are often formulated as salts to enhance their solubility under physiological conditions. Examples of alkaloid salt counter ions include the appropriate counter ion derived from but in no way limited to mineral acids such as hydrochloric acid and sulfuric acid as well as organic acid counter ions including but not limited to tartaric acid and maleic acid.

As used herein, “epimers” refers to diastereomers that differ in configuration of only one stereogenic center. Diastereomers are a class of stereoisomers that are non-superposable, non-mirror images of one another, unlike enantiomers that are non-superposable mirror images of one another. The current invention considers specific stereoisomers as described by the structures.

As used herein, “absolute stereoisomer” refers to a very specific single enantiomer with a specific configuration, which is often indicated by a particular structure.

As used herein, the term “salts” refers to any salt that complexes with identified compounds contained herein while retaining a desired function, e.g., biological activity. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid.

As used herein, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “hydroxyamino” means —NHOH; “nitro” means —NO₂; imino means ═NH (see below for definitions of groups containing the term imino, e.g., alkylamino); “cyano” means —CN; “azido” means —N₃; “mercapto” means —SH; “thio” means ═S; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); and “silyl” means —SiH₃ (see below for definitions of group(s) containing the term silyl, e.g., alkylsilyl).

For the groups below, the following parenthetical subscripts further define the groups as follows: “(Cn)” defines the exact number (n) of carbon atoms in the group; “(C≦n)” defines the maximum number (n) of carbon atoms that can be in the group; (Cn-n′) defines both the minimum (n) and maximum number (n′) of carbon atoms in the group. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3-10 carbon atoms)). Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3-10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CM₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two σ-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

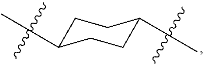 [SMILES: *C1CCC(*)CC1 ; ","]

are non-limiting examples of alkanediyl groups. The term “substituted alkanediyl” refers to a non-aromatic monovalent group, wherein the alkynediyl group is attached with two 6-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

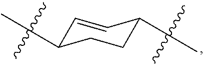 [SMILES: *C1C=CC(*)CC1 ; ","]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CCH₃, —C≡CCH₃, —C≡CC₆H₅ and —CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “aryl” when used without the “substituted” modifier refers to a monovalent group with an aromatic carbon atom as the point of attachment, said carbon atom forming part of a six-membered aromatic ring structure wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of a six-membered aromatic ring structure wherein the ring atoms are all carbon, and wherein the monovalent group further has at least one atom independently selected from the group consisting of N, O, F, Cl, Br, I, Si, P, and S, Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

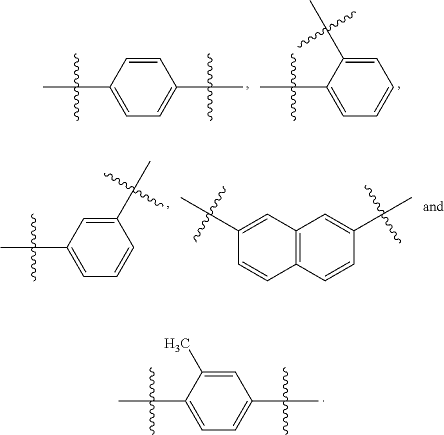

The term “substituted arenediyl” refers to a divalent group, wherein the arenediyl group is attached with two a-bonds, with two aromatic carbon atoms as points of attachment, said carbon atoms forming part of one or more six-membered aromatic rings structure(s), wherein the ring atoms are all carbon, and wherein the divalent group further has at least one atom independently selected from the group consisting of N, O, F, Cl, Br, I, Si, P, and S.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

The term “heteroarenediyl” when used without the “substituted” modifier refers to a divalent group, wherein the heteroarenediyl group is attached with two a-bonds, with an aromatic carbon atom or nitrogen atom as the point of attachment, said carbon atom or nitrogen atom two aromatic atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of heteroarenediyl groups include:

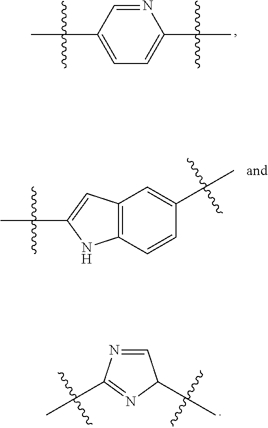 [SMILES: *c1ccc(*)nc1 ; "," ; "and" ; *c1ccc2[nH]c(*)cc2c1 ; *C1=NC(*)C=N1 ; "."]

The term “substituted heteroarenediyl” refers to a divalent group, wherein the heteroarenediyl group is attached with two a-bonds, with two aromatic carbon atoms as points of attachment, said carbon atoms forming part of one or more six-membered aromatic rings structure(s), wherein the ring atoms are all carbon, and wherein the divalent group further has at least one atom independently selected from the group consisting of N, O, F, Cl, Br, I, Si, P, and S.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, ═CH(CH₂CH₃), and ═C(CH₃)₂. The term “substituted alkylidene” refers to the group ═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that teitu is defined above. For example, —OCH₂CF₃ is a substituted alkoxy group.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one a-bond and one n-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, —C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, S(O)₂-cyclopentyl, and —S(O)₂-cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or —NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)+, —NH₂(CH₂CH₂CH₃)+, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, —N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or —NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′⁺, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, SH(cyclopentyl)⁺, and —SH(cyclohexyl)⁺. The term “substituted alkylsulfonium” refers to the group —SRR′⁺, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺ is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A compound having a formula that is represented with a dashed bond is intended to include the formulae optionally having zero, one or more double bonds. Thus, for example, the structure

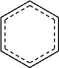

includes the structures

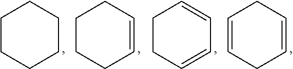

and

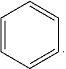

As will be understood by a person of skill in the art, no one such ring atom forms part of more than one double bond.

Any undefined valency on an atom of a structure shown in this application implicitly represents a hydrogen atom bonded to the atom.

A ring structure shown with an unconnected “R” group, indicates that any implicit hydrogen atom on that ring can be replaced with that R group. In the case of a divalent R group (e.g., oxo, imino, thio, alkylidene, etc.), any pair of implicit hydrogen atoms attached to one atom of that ring can be replaced by that R group. This concept is as exemplified below:

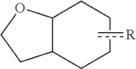

represents

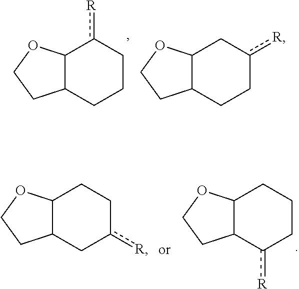

As used herein, a “chiral auxiliary” refers to a removable chiral group that is capable of influencing the stereoselectivity of a reaction. Persons of skill in the art are familiar with such compounds, and many are commercially available.

The term “protecting group,” as that term is used in the specification and/or claims, is used in the conventional chemical sense as a group, which reversibly renders unreactive a functional group under certain conditions of a desired reaction and is understood not to be H. After the desired reaction, protecting groups may be removed to deprotect the protected functional group. All protecting groups should be removable (and hence, labile) under conditions which do not degrade a substantial proportion of the molecules being synthesized. In contrast to a protecting group, a “capping group” permanently binds to a segment of a molecule to prevent any further chemical transformation of that segment. It should be noted that the functionality protected by the protecting group may or may not be a part of what is referred to as the protecting group.

Protecting groups include but are not limited to: Alcohol protecting groups: Acetoxy group, β-Methoxyethoxymethyl ether (MEM), methoxymethyl ether (MOM), p-methoxybenzyl ether (PMB), methylthiomethyl ether, pivaloyl (Piv), tetrahydropyran (THP), silyl ethers (including but not limited to trimethylsilyl (TMS), tert-butyldimethylsilyl (TBDMS), and triisopropylsilyl (TIPS) ethers), methyl ethers, and ethoxyethyl ethers (EE). Amine protecting groups: carbobenzyloxy (Cbz) group, p-methoxybenzyl carbonyl (Moz or MeOZ) group, tert-butyloxycarbonyl (BOC) group, 9-fluorenylmethyloxycarbonyl (FMOC) group, benzyl (Bn) group, p-methoxybenzyl (PMB), dimethoxybenzyl (DMPM), p-methoxyphenyl (PMP) group, tosyl (Ts) group, and other sulfonamides (Nosyl & Nps) groups. Carbonyl protecting groups: acetals, ketals, acylals, and dithianes. Carboxylic acid protecting groups: alkyl esters, aryl esters, silyl esters. Protection of terminal alkynes protected as propargyl alcohols in the Favorskii reaction.

The teen “leaving group,” as that term is used in the specification and/or claims, is an atom or group (charged or uncharged) that becomes detached from an atom in what is considered to be the residual or main part of the substrate in a specified reaction.

Leaving groups include, but are not limited to: NH₂⁻ (amine), CH₃O⁻ (methoxy), HO⁻ (hydroxyl), CH₃COO⁻ (carboxylate), H₂O (water), F⁻, Cl⁻, Br⁻, I⁻, N₃⁻ (azide), SCN⁻ (thiocyanate), NO₂ (nitro), and protecting groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use (P. H. Stahl & C. G. Wermuth eds., Verlag Helvetica Chimica Acta, 2002) [5].

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methane-sulfonates, ethanesulfonates, benzenesulfonates, p-toluenesulfonates, cyclohexyl-sulfamates, quinates, esters of amino acids, and the like. Similarly, a compound comprising an amine group may be administered as an amide that is converted by hydrolysis in vivo to the amine compound.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

Enantiomers are compounds that individually have properties said to have “optical activity” and consist of chiral molecules. If a chiral molecule is dextrorotary, its enantiomer will be levorotary, and vice-versa. In fact, the enantiomers will rotate polarized light the same number of degrees, but in opposite directions. “Dextrorotation” and “levorotation” (also spelled laevorotation) refer, respectively, to the properties of rotating plane polarized light clockwise (for dextrorotation) or counterclockwise (for levorotation). A compound with dextrorotation is called “dextrorotary,” while a compound with levorotation is called “levorotary”.

A standard measure of the degree to which a compound is dextrorotary or levorotary is the quantity called the “specific rotation” “[α]”. Dextrorotary compounds have a positive specific rotation, while levorotary compounds have negative. Two enantiomers have equal and opposite specific rotations. A dextrorotary compound is prefixed “(+)-” or “d-”. Likewise, a levorotary compound is often prefixed “(−)-” or “l-”. These “d-” and “l-” prefixes should not be confused with the “D-” and “L-” prefixes based on the actual configuration of each enantiomer, with the version synthesized from naturally occurring (+)-compound being considered the D-form. A mixture of enantiomers of the compounds is prefixed “(±)-”. An equal mixture of enantiomers of the compounds is considered “optically inactive”.

When used herein, unless otherwise specified, “morphine” refers to a mixture of enantiomers of morphine, “(±)-morphine.” When used herein, unless otherwise specified, codiene refers to a mixture of enantiomers of codeine, “(±)codeine,” or a single enantiomer, e.g. “(−)-codeine.”

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like.

The present invention contemplates the above-described compositions in “therapeutically effective amounts” or “pharmaceutically effective amounts”, which means that amount which, when administered to a subject or patient for treating a disease, is sufficient to effect such treatment for the disease or to emeliorate one or more symptoms of a disease or condition (e.g. emeliorate pain).

The above definitions supersede any conflicting definition in any of the reference that is incorporated by reference herein.

The present invention contemplates, in certain embodiments inhibiting or preventing disease (e.g. treating early Alzheimer's with galanthamine). As used herein, the terms “prevent” and “preventing” include the prevention of the recurrence, spread or onset of a disease or disorder. It is not intended that the present invention be limited to complete prevention. In some embodiments, the onset is delayed, or the severity of the disease or disorder is reduced. Studies with galanthamine have showed mild cognitive and global benefits for patients with Alzheimer's disease.

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

“Subject” refers to any mammal, preferably a human patient, livestock, or domestic pet.

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

The term “salts”, as used herein, refers to any salt that complexes with identified compounds contained herein while retaining a desired function, e.g., biological activity. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Pharmaceutically acceptable salts also include base addition salts, which may be formed when acidic protons present are capable of reacting with inorganic or organic bases. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, and trimethylamine. All of these salts may be prepared by conventional means from the corresponding compound of the invention by reacting, for example, the appropriate acid or base with the compound of the invention. Unless otherwise specifically stated, the present invention contemplates pharmaceutically acceptable salts of the considered pro-drugs.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

In structures wherein stereochemistry is not explicitly indicated, it is assumed that all stereochemistry is considered and all isomers claimed. In structures where the specific isomers or enantiomers are indicated, the specific enantiomer is claimed.

Any undefined valency on an atom of a structure shown in this application implicitly represents a hydrogen atom bonded to the atom. Bonds to copper (Cu) metal may be coordinate bonds and are not necessarily considered covalent.

The term “effective,” as that term is used in the specification and/or claims, means adequate to accomplish a desired, or hoped for result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

The term “pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts, which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts Properties, and Use (P. H. Stahl & C. G. Wermuth eds., Verlag Helvetica Chimica Acta, 2002) [5] herein incorporated by reference. Unless otherwise specifically stated, the present invention contemplates pharmaceutically acceptable salts of the considered pro-drugs.

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

The term“prevention” or “preventing” as used herein includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers.

Enantiomers are compounds that individually have properties said to have “optical activity” and consist of molecules with at least one chiral center, almost always a carbon atom. If a particular compound is dextrorotary, its enantiomer will be levorotary, and vice-versa. In fact, the enantiomers will rotate polarized light the same number of degrees, but in opposite directions. “Dextrorotation” and “levorotation” (also spelled laevorotation) refer, respectively, to the properties of rotating plane polarized light clockwise (for dextrorotation) or counterclockwise (for levorotation). A compound with dextrorotation is called “dextrorotary,” while a compound with levorotation is called “levorotary.”

A standard measure of the degree to which a compound is dextrorotary or levorotary is the quantity called the “specific rotation” “[α]”. Dextrorotary compounds have a positive specific rotation, while levorotary compounds have negative. Two enantiomers have equal and opposite specific rotations. A dextrorotary compound is prefixed “(+)-” or “d-”. Likewise, a levorotary compound is often prefixed “(−)” or “l-”. These “d-” and “l-” prefixes should not be confused with the “D-” and “L-” prefixes based on the actual configuration of each enantiomer, with the version synthesized from naturally occurring (+)-compound being considered the D-form. A mixture of enantiomers of the compounds is prefixed “(±)-”. An equal mixture of enantiomers of the compounds is considered “optically inactive.”

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

The present invention contemplates the above-described compositions in “therapeutically effective amounts” or “pharmaceutically effective amounts”, which means that amount which, when administered to a subject or patient for treating a disease, is sufficient to effect such treatment for the disease or to ameliorate one or more symptoms of a disease or condition (e.g. ameliorate pain).

As used herein, the terms “treat” and “treating” are not limited to the case where the subject (e.g. patient) is cured and the disease is eradicated. Rather, the present invention also contemplates treatment that merely reduces symptoms, improves (to some degree) and/or delays disease progression. It is not intended that the present invention be limited to instances wherein a disease or affliction is cured. It is sufficient that symptoms are reduced.

“Subject” refers to any mammal, preferably a human patient, livestock, or domestic pet.

In a specific embodiment, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient or vehicle with which the active compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a subject, the pharmaceutically acceptable vehicles are preferably sterile. Water can be the vehicle when the active compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The present compositions, if desired, can also contain minor amounts of wetting or emulsifying agents, or pH buffering agents.

Pharmaceutically acceptable sugars include but are not limited to sucrose, dextrose, maltose, galactose, rhamnose, and lactose. Pharmaceutically acceptable sugar alcohols include but are not limited to mannitol, xylitol, and sorbitol.

As used herein, “extended release” refers to providing continuous therapeutic level of an active agent (e.g., neuregulin) over a period of time. The extended release includes, without limitation various forms of release, such as continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slow acting, sustained action, sustained-action medications, and controlled release. The ability to obtain extended release, controlled release, timed release, sustained release, delayed release, long acting, pulsatile delivery or immediate release is performed using well-known procedures and techniques available to the ordinarily skilled artisan.

The amount of time over which the active agent continues to be released depends on the characteristics of the active agent and the extended release technology or technologies used, but in all cases is longer than that of administration of the active agent without the extended release technology or technologies. Other forms of slow release compositions are described in the following: U.S. Pat. No. 4,828,836 [6], U.S. Pat. No. 6,190,591 [7].

### DETAILED DESCRIPTION OF THE INVENTION

The present invention relates to methods for the synthesis of morphine and derivatives thereof. In preferred embodiments, the invention relates to methods for improving the efficiency and overall yield of said morphine and derivatives. It is not intended that the present invention be limited to any particular chemical, biochemical or biological mechanism or theory.

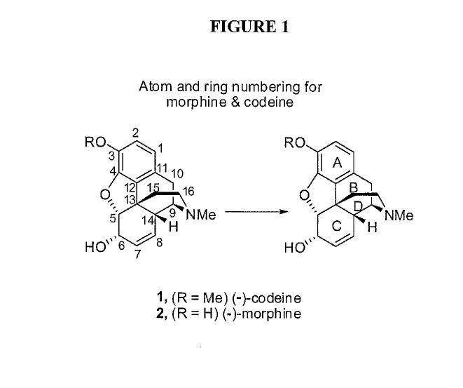

The importance of morphine 1, codeine 2 (FIG. 1) and various derivatives as central drugs for the control of pain management cannot be over estimated. They, and various derivatives, have occupied the central place in analgesia and anesthetics for over 100 years, even before their structures were known. Despite this, there are well known side effects associated with these drugs such as respiratory depression, nausea, vomiting, physical dependence and addiction. Consequently, there continues to be a need for new analogues of these compounds that have less side effects. In this application, the synthesis of simple new derivatives that have potentially valuable biological properties are described.

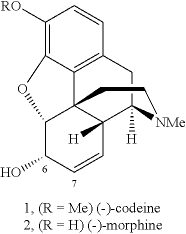

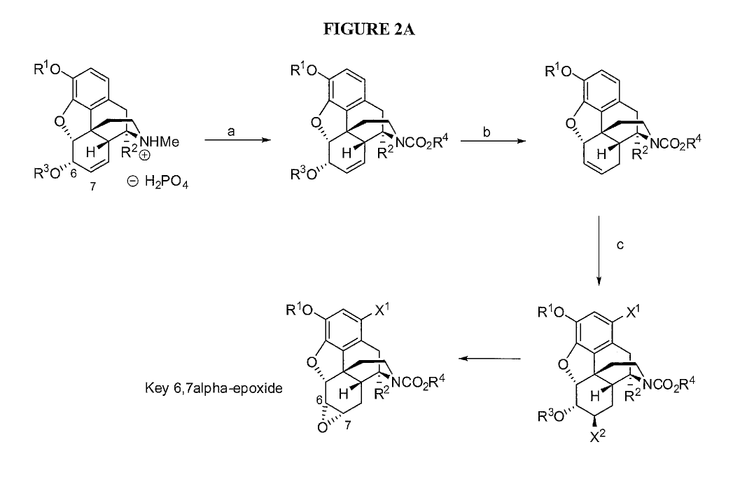

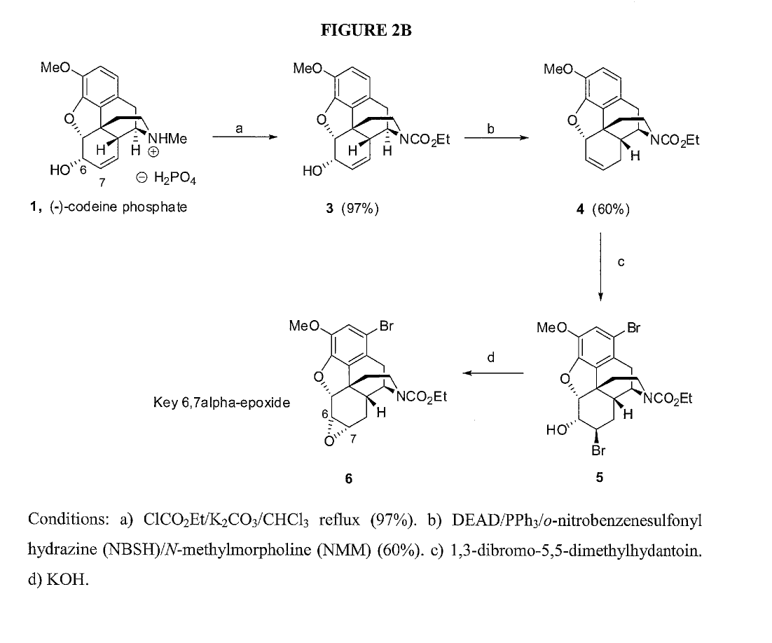

Starting with (−)-codeine phosphate 1, it was converted into the known carbamate 3 following literature procedures (FIG. 2B) [2]. Treatment of 3 carbamate with DEAD/PPh₃/NMM/NBSH [3] gave the 6,7-alkene 4 as a single enantiomer (see FIG. 2B). This compound was previously made by total synthesis as a racemate [4]. Access to compound 4 through synthesis from codeine is much shorter, and supplies material that can be converted into derivatives for biological assays that are single optical isomers.

Conditions: a) ClCO₂Et/K₂CO₃/CHCl₃ reflux (97%). b) DEAD/PPh₃/o-nitrobenzenesulfonyl hydrazine (NBSH)/N-methylmorpholine (NMM) (60%). c) 1,3-dibromo-5,5-dimethylhydantoin. d) KOH. (see FIG. 2B)

The alkene 4 was converted into the epoxide 6 via the bromohydrin 5 using our previous method that involves treatment of 4 with 1,3-dibromo-5,5-dimethylhydantoin to give 5, which was treated with KOH resulting in the 6,7α-epoxide 6 (FIG. 2B) [4]. This route provides a concise method for making the key derivative 6 as a single enantiomer from (−)-codeine, rather than the previously reported method by total synthesis in a racemic form [4, 8].

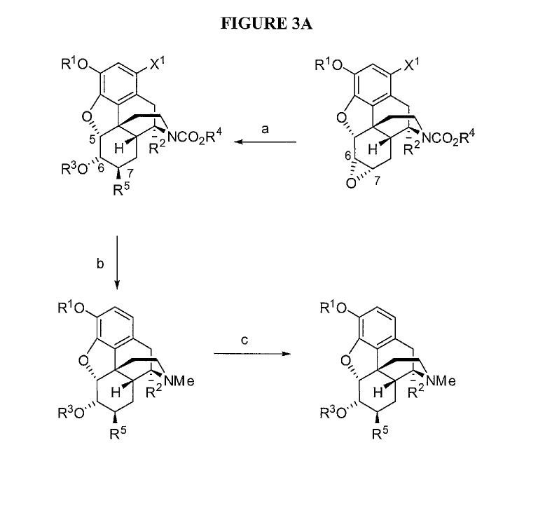

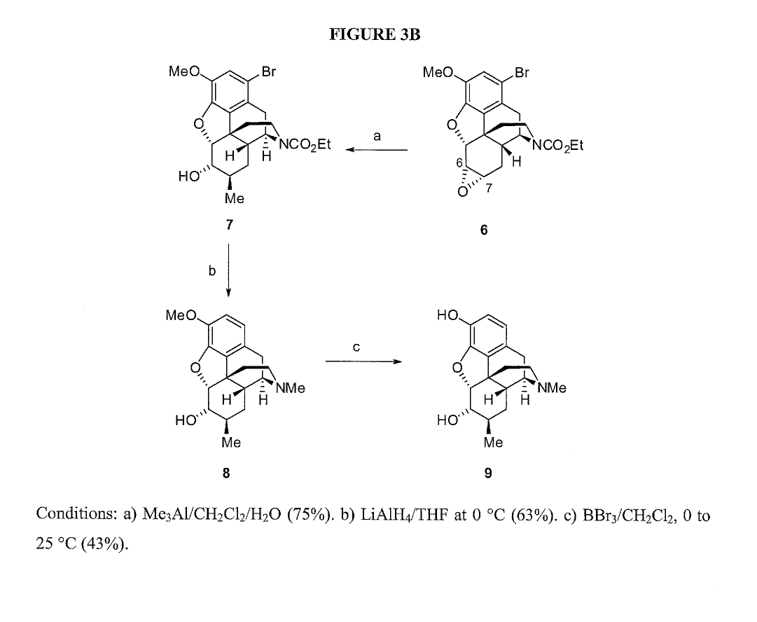

Treatment of 6 with Me₃Al/PhMe/H₂O gave 7 in 75% yield. Reduction of 7 with LiAlH₄ resulted in 8, which when exposed to BBr₃/CH₂Cl₂ gave 9 (FIG. 3B). Both 8 and 9 exhibited increased potency when compared with codeine and morphine respectively. The reaction conditions included: a) Me₃Al/CH₂Cl₂/H₂O (75%); b) LiAlH₄/THF at 0° C. (63%); and c) BBr₃/CH₂Cl₂, 0 to 25° C. (43%).

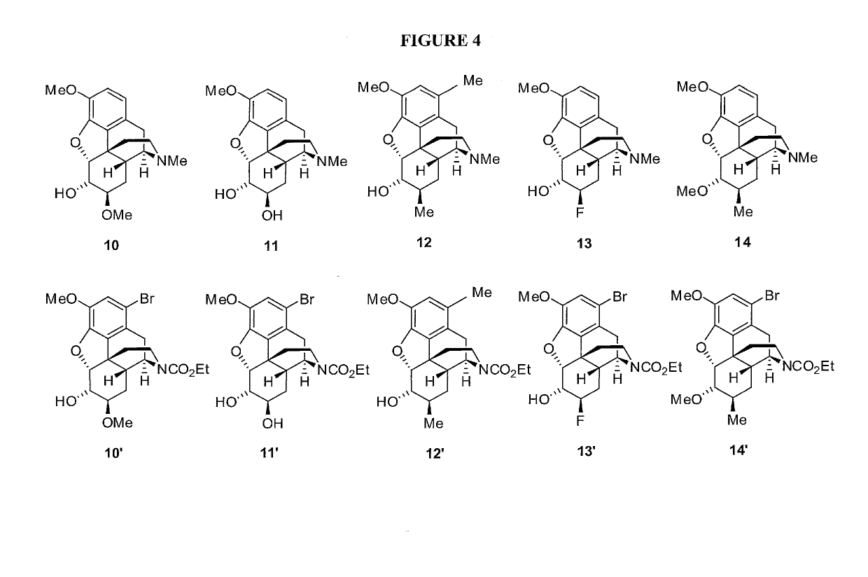

FIG. 4 provides a several specific (non-limiting) examples of additional morphine and codeine derivatives, compounds 10, 10′, 11, 11′, 12, 12′, 13, 13′, 14 and 14′.

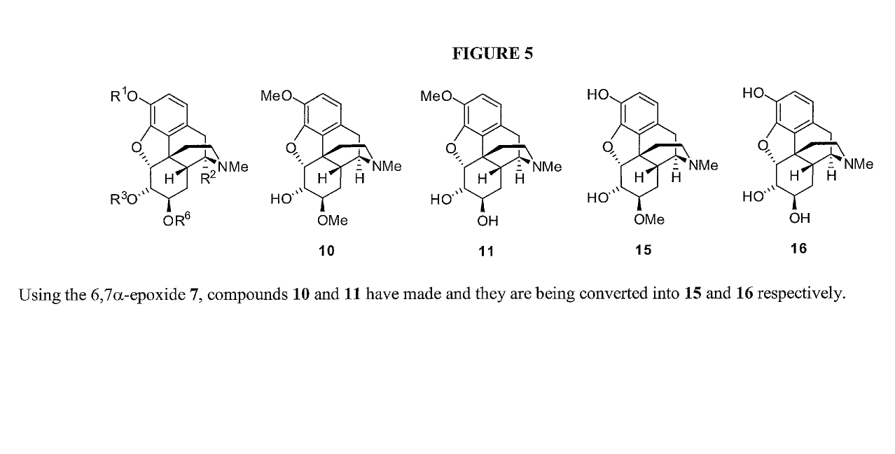

Using the 6,7α-epoxide 7, the compounds 10 and 11 (FIG. 5) have been made and they are being converted into 15 and 16 respectively.

In preferred embodiments, the invention relates to methods and compositions comprising morphine and derivatives thereof. Morphine ((5α,6α)-7,8-didehydro-4,5-epoxy-17-methylmorphinan-3,6-diol; C₁₇H₁₉NO₃; MW=285.4), a member of the alkaloid class of compounds, is a highly effective analgesic used in a myriad of pharmaceutical and biomedical applications. While there are numerous reported synthetic strategies for obtaining limited quantities and percent yields of morphine alkaloids such as Zezula et al. (2007) Synlett, 2863-2867 [9]; Omori et al. (2007) Synlett, 2859-2862 [10]; Uchida et al. (2006) Org. Lett. 8, 5311-5313 [11] and Trost et al. (2005) J. Am. Chem. Soc. 127, 14785-14803 [12], all of which are hereby incorporated by reference, one of the most practical synthetic strategies for obtaining opium alkaloids is the Rice adaptation of the Grewe strategy as provided for in Rice (1980) J. Org. Chem. 45, 3135-3137 [13], hereby incorporated by reference. Other advancements in the synthesis of morphine and related derivatives are described in Magnus et al. “Efficient Synthesis of Morphine and Codeine,” U.S. patent application Ser. No. 12/778,466, filed May 12, 2010 [8]. While not limiting the scope of the current invention, the biosynthetic steps utilized by nature for the generation of morphine alkaloids is well understood.

### Pharmaceutical Formulations

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155 [14], hereby incorporated by reference).

In a preferred embodiment, the active compound and optionally another therapeutic or prophylactic agent are formulated in accordance with routine procedures as pharmaceutical compositions adapted for intravenous administration to human beings. Typically, the active compounds for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the active compound is to be administered by infusion, it can be dispensed, for example, with an infusion bottle containing sterile pharmaceutical grade water or saline. Where the active compound is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an orally administered of the active compound. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the active compound can be delayed or prolonged by proper formulation. For example, a slowly soluble pellet of the active compound can be prepared and incorporated in a tablet or capsule. The technique can be improved by making pellets of several different dissolution rates and filling capsules with a mixture of the pellets. Tablets or capsules can be coated with a film that resists dissolution for a predictable period of time. Even the parenteral preparations can be made long acting, by dissolving or suspending the compound in oily or emulsified vehicles, which allow it to disperse only slowly in the serum.

Compositions for use in accordance with the present invention can be formulated in conventional manner using one or more physiologically acceptable carriers or excipients.

Thus, the compound and optionally another therapeutic or prophylactic agent and their physiologically acceptable salts and solvates can be formulated into pharmaceutical compositions for administration by inhalation or insufflation (either through the mouth or the nose) or oral, parenteral or mucosol (such as buccal, vaginal, rectal, sublingual) administration. In some embodiments, the administration is optical (e.g. eyes drops applied directly to the eye). In one embodiment, local or systemic parenteral administration is used.

For oral administration, the compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

In addition to the formulations described previously, the compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician's Desk Reference (62^{nd} ed. 2008, herein incorporated by reference in its entirety) [15].

Methods of administering the active compound and optionally another therapeutic or prophylactic agent include, but are not limited to, parenteral administration (e.g., intradermal, intramuscular, intraperitoneal, intravenous and subcutaneous), epidural, and mucosal (e.g., intranasal, rectal, vaginal, sublingual, buccal or oral routes). In a specific embodiment, the active compound and optionally another prophylactic or therapeutic agents are administered intramuscularly, intravenously, or subcutaneously. The active compound and optionally another prophylactic or therapeutic agent can also be administered by infusion or bolus injection and can be administered together with other biologically active agents. Administration can be local or systemic. The active compound and optionally the prophylactic or therapeutic agent and their physiologically acceptable salts and solvates can also be administered by inhalation or insufflation (either through the mouth or the nose). In a preferred embodiment, local or systemic parenteral administration is used.

In specific embodiments, it can be desirable to administer the active compound locally to the area in need of treatment. This can be achieved, for example, and not by way of limitation, by local infusion during surgery or topical application, e.g., in conjunction with a wound dressing after surgery, by injection, by means of a catheter, by means of a suppository, or by means of an implant, said implant being in one embodiment of a porous, non-porous, or gelatinous material, including membranes, such as silastic membranes, or fibers.

Pulmonary administration can also be employed, e.g., by use of an inhaler or nebulizer, and formulation with an aerosolizing agent, or via perfusion in a fluorocarbon or synthetic pulmonary surfactant. In certain embodiments, the active compound can be formulated as a suppository, with traditional binders and vehicles such as triglycerides.

Selection of a particular effective dose can be determined (e.g., via clinical trials) by a skilled artisan based upon the consideration of several factors, which will be known to one skilled in the art. Such factors include the disease to be treated or prevented, the symptoms involved, the subject's body mass, the subject's immune status and other factors known by the skilled artisan.

The dose of the active compound to be administered to a subject, such as a human, is rather widely variable and can be subject to independent judgment. It is often practical to administer the daily dose of the active compound at various hours of the day. However, in any given case, the amount of the active compound administered will depend on such factors as the solubility of the active component, the formulation used, subject condition (such as weight), and/or the route of administration.

Thus, specific compositions and methods of chemical transformations of (−)-codeine to afford derivatives of codeine and morphine thereof have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### EXAMPLES

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); TLC (thin layer chromatography).

All reactions were setup under an atmosphere of argon and only degassed when specified. Melting points were taken on a Thomas-Hoover capillary melting point apparatus and are uncorrected Infrared spectra were recorded on a Nicolet FT-IR spectrophotometer neat on a KBr plate unless otherwise indicated. ¹H NMR spectra were recorded on a Varian spectrometer at 300 MHz, or 500 MHz, or 600 MHz in the indicated solvent and are reported in ppm relative to tetramethylsilane and referenced internally to the residually protonated solvent. ¹³C NMR spectra were recorded on at 75 MHz, or 125 MHz, or 150 MHz in the solvent indicated and are reported in ppm relative to tetramethylsilane and referenced internally to the residually protonated solvent. Mass spectra were obtained on a VG ZAB2E or a Finnigan TSQ70. Routine monitoring of reactions was performed using Merck 60 F₂₅₄ silica gel, aluminum-backed TLC plates. Flash column chromatography was performed using EMD silica gel (particle size 0.040-0.063 μm 22×250 mm) Solvents and commercial reagents were purified in accordance with Perrin and Armarego [16] or used without further purification.

### Example 1

### N-Desmethyl-N-carboethoxycodeine (3)

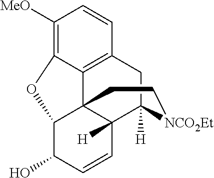

As shown in FIG. 2 (the specific route from 1 to 3), Ethyl chloroformate (5.8 mL, 60.4 mmol) was added to a mixture of codeine phosphate 1 (4 g, 10.07 mmol) and potassium carbonate (8.4 g, 60.4 mmol) in chloroform (300 mL), and the mixture was heated at reflux under argon while stirred. The reaction was monitored by thin layer chromatography (100% ethyl acetate; anisaldehyde stain). Upon completion (24 h), the mixture was cooled to 25° C., diluted with chloroform (100 mL), and washed with water (3×75 mL), followed by brine (50 mL of saturated NaCl), dried (Na₂SO₄), and concentrated in vacuo. The crude product was purified by chromatography over silica gel eluting with EtOAc/hexanes from 0% to 75% EtOAc/hexanes to give 3 (3.5 g, 97% yield) as white amorphous solid. ¹H NMR (300 MHz, CDCl₃) 6.62 (1H, d, J=8.1 Hz), 6.50 (1H, d, J=7.8 Hz), 5.71 (1H, d, J=9.3 Hz), 5.24 (1H, d, J=9.6 Hz), 4.90 (0.6H, bs), 4.77 (0.4H, bs), 4.82 (1H, d, J=6 Hz), 4.12 (2H, q, J=6.9 Hz), 4.06-3.95 (2H, m), 3.74 (3H, s), 3.28 (1H, bs), 3.03-2.82 (1H, m), 2.78 (1H, d, J=6.3 Hz), 2.67 (1H, d, J=18.6 Hz), 2.48 (1H, bs), 1.94-1.78 (2H, m), 1.22 (3H, t, J's=7.2 and 8.4 Hz). ¹³C NMR (75 MHz, CDCl₃) δ 155.6, 146.8, 142.6, 134.8, 130.8, 127.1, 126.1, 120.1, 113.5, 91.5, 66.4, 61.7, 56.4, 50.5, 43.6, 40.0, 37.4, 35.5, 29.6, 15.0.

### Example 2

### N-Desmethyl-N-carboethoxy-6-deoxy-7,8-dihydro codeine-6,7-ene (4)

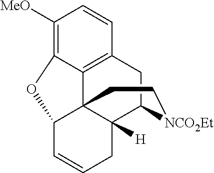

As shown in FIG. 2B, diethyl azodicarboxylate (0.61 mL (3.9 mmol) was added to a solution of triphenylphosphine (1.1 g 4.2 mmol) in N-methylmorpholine (16 mL) under argon at −30° C. The mixture was stirred for 10 min, followed by the addition of 3 (0.58 g, 1.62 mmol). The solution was stirred for 60 min at −3° C., and o-nitrobenzenesulfonyl hydrazine (0.85 g, 3.9 mmol) (NBSH) was added to the reaction at −30° C., the mixture was warmed to room temperature (25° C.) while stirring. The reaction was monitored by thin layer chromatography (60% EtOAc/hexanes, anisaldehyde stain). Upon completion (2 h), the mixture was diluted with EtOAc (50 mL), washed with water (3×25 mL), followed by a wash with brine (25 mL, saturated NaCl), dried (Na₂SO₄), and concentrated in vacuo. The crude product was purified by chromatography eluting with EtOAc/hexanes from 0% to 10% EtOAc/hexanes to give 4 (330 mg, 60% yield) as clear oil. IR (thin film) 2978, 2931, 2838, 1695 cm⁻¹. ¹H NMR (300 MHz, CDCl₃) δ 6.73 (1H, d, J=8 Hz), 6.62 (1H, d, J=8 Hz), 5.85 (1H, m), 5.71 (1H, d, J=10 Hz), 4.95 (1H, s), 4.71 (0.6H, bs), 4.56 (0.4H, bs), 4.15 (2H, q, J=7.2 Hz), 4.10-3.93 (1H, m), 3.85 (3H, s), 3.06-2.86 (2H, m), 2.68 (1H, d, J=18 Hz), 2.30-2.25 (1H, m), 2.0 (1H, dt, J's=6 and 10 Hz), 1.90-1.70 (2H, m), 1.53-1.40 (1H, m), 1.29-1.18 (3H, m). ¹³C NMR (75 MHz, CDCl₃) δ 155.4, 144.9, 143.5, 131.9, 128.6, 125.9, 124.7, 119.0, 113.3, 87.4, 61.4, 56.3, 50.2, 41.2, 37.9, 37.8, 35.1, 28.9, 24.1, 14.7.

### Example 3

### Compounds 5 and 6

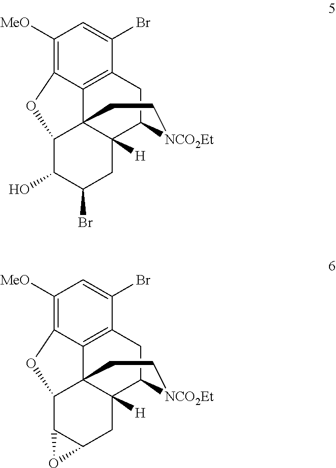

As shown in FIG. 2B, to a stirred solution of 4 (330 mg, 0.97 mmol) in dioxane (20 mL) and water (20 mL) at −10° C. was added 1,3-dibromo-5,5-dimethylhydantoin (555 mg, 1.94 mmol) in the dark. The mixture was stirred, and warmed to room temperature for 12 h to give compound 5. The solution of 5 was directly treated with KOH (220 mg, 3.9 mmol) heated at 75° C. The reaction was monitored by thin layer chromatography (30% ethyl acetate/hexane, anisaldehyde stain). Upon completion (24 h), the reaction was diluted with EtOAc (50 mL), washed with water (3×25 mL), followed by a wash with brine (25 mL, saturated NaCl), dried (Na₂SO₄), and concentrated in vacuo. The crude product was purified by chromatography eluting with (0-30% EtOAc/hexanes) to give 6 (360 mg, 50% yield) as an off-white amorphous solid.

Compound 5. IR (thin film) 3420, 2978, 2937, 2889, 1684 cm⁻¹. ¹H NMR (300 MHz, CDCl₃) δ 6.95 (1H, s), 4.90 (1H, d, J=5.1 Hz), 4.74 (0.6H, bs), 4.59 (0.4H, bs), 4.28-4.10 (3H, q, J=7.2 Hz), 4.10-3.94 (1H, m), 3.87 (3H, s), 2.84-2.48 (5H, m), 1.91-1.62 (4H, m), 1.35-1.25 (4H, m). ¹³C NMR (75 MHz, CDCl₃) δ 155.6, 146.0, 143.1, 130.6, 125.3, 116.9, 113.2, 88.2, 70.4, 62.0, 56.7, 50.6, 50.3, 42.5, 38.0, 36.9, 36.1, 30.4, 26.7, 14.9. HRMS: C₂₀H₂₃Br₂NO₅ requires (M+H) calc. 516.0021, found 516.0018.

Compound 6. IR (thin film) 2963, 2926, 2850, 1695, 1684 cm⁻¹. ¹H NMR (300 MHz, CDCl₃) δ 6.91 (1H, s), 4.87 (1H, d, J=3.7 Hz), 4.70 (0.7H, bs), 4.56 (0.3H, bs), 4.13 (2H, q, J=7.3 Hz), 4.10-3.89 (1H, m), 3.86 (3H, s), 3.30-3.23 (2H, m), 2.79-2.70 (2H, m), 2.53 (1H, d, J=18 Hz), 2.00 (2H, m), 1.79-1.68 (2H, m), 1.27 (3H, t, J=7.2 Hz), 1.15-1.06 (1H, m). ¹³C NMR (75 MHz, CDCl₃) δ 155.2, 146.1, 143.0, 129.3, 124.3, 116.8, 112.1, 87.7, 61.6, 56.5, 53.6, 50.9, 50.2, 49.9, 41.2, 37.4, 37.2, 30.1, 23.0, 14.7. HRMS: C₂₀H₂₂BrNO₅ requires (M+H) calc. 436.0760, found 436.0758.

### Example 4

### Compound 7

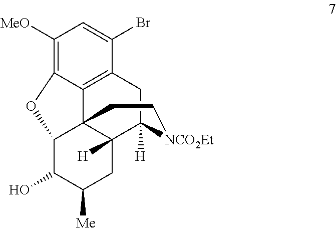

As shown in FIG. 3B, to a solution of 6 (160 mg, 0.37 mmol) in dichloromethane (4 mL) and water (0.1 mL) was slowly added with trimethylaluminum (3.7 mL, 7.4 mmol in hexane 2M). Once the gas evolution subsided, the reaction was monitored by thin layer chromatography (10% EtOAc/dichloromethane, anisaldehyde stain). Upon completion, the reaction was cooled to 0° C. and slowly quenched with water (5 mL), followed by 30% Rochelle's salt (5 mL). The reaction was extracted with EtOAc (3×10 mL), dried (Na₂SO₄) and concentrated in vacuo. The crude product was purified by chromatography (0-10% EtOAc/dichloromethane) to give 7 (128 mg, 80% yield) as an off-white, amorphous solid. IR (thin film) 3439, 2934, 1700, 1684, 1489, 1433 cm⁻¹. ¹H NMR (300 MHz, CDCl₃) δ 6.95 (1H, s), 4.71 (1H, d, J=4.5 Hz), 4.66 (0.5H, bs), 4.56 (0.5H, bs), 4.21-4.10 (3H, m), 4.08-3.90 (2H, m), 3.86 (3H, s), 3.52-3.49 (1H, m), 2.90-2.70 (2H, m), 2.58 (1H, d, J=18.6 Hz), 2.20-2.10 (2H, m), 1.88-1.68 (2H, m), 1.50-1.10 (4H, m), 0.91 (3H, d, J=5.4 Hz). ¹³C NMR (75 MHz, CDCl₃) δ 155.5, 146.2, 143.1, 131.4, 126.0, 116.8, 112.9, 91.3, 72.4, 61.8, 56.7, 50.3, 43.7, 37.4, 37.3, 35.3, 30.6, 28.8, 26.6, 18.3, 14.6. HRMS: C₂₁H₂₆BrNO₅ requires (M) calc. 451.0994, found 451.0992.

### Example 5

### 7β-Methyl-7,8-dihydrocodeine (8)

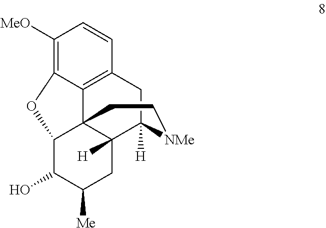

As shown in FIG. 3B, to a stirred solution of 7 (100 mg, 0.22 mmol) in tetrahydrofuran (5 mL) under argon at 0° C. was slowly added 2M lithium aluminum hydride in THF (1 mL, 1.9 mmol). The reaction was monitored by thin layer chromatography (5% MeOH in dichloromethane, anisaldehyde stain). Upon completion, as judged by TLC, the reaction was slowly quenched, first with MeOH (2 mL) and then Rochelle's salt (2 mL). The mixture was extracted with 5% MeOH in CHCl₃ (3×10 mL), dried (Na₂SO₄), and concentrated in vacuo. The crude product was purified by chromatography (0-5% MeOH/diehloromethane) to give 8 (44 mg (63%) yield as an off-white, amorphous solid. The final product 8 was converted to its HCl salt form by stirring it in 5 mL of 1N HCl in methanol, followed by concentration in vacuo. IR (thin film) 3600, 3442, 2932, 1653, 1601, 1499, 1452 cm⁻¹. ¹H NMR (300 MHz, CDCl₃) δ 6.69 (1H, d, J=8.1 Hz), 6.58 (1H, d, J=8.4 Hz), 4.66 (1H, d, J=4.8 Hz), 3.82 (3H, s), 3.45 (1H, m), 3.03 (1H, m), 2.94 (1H, d, J=18.6 Hz), 2.50-2.18 (5H, m), 2.35 (3H, s), 1.95-1.85 (1H, dt, J=5.1 Hz), 1.68 (1H, d, J=12.6 Hz), 1.26-1.11 (3H, m), 0.84 (3H, d, J=6 Hz). ¹³C NMR (75 MHz, CDCl₃) δ 146.5, 141.9, 131.0, 127.7, 119.4, 113.7, 91.5, 72.9, 59.9, 56.7, 46.5, 43.3, 42.9, 37.6, 36.1, 29.8, 29.5, 20.1, 18.7. HRMS: C₁₉H₂₅NO₃ requires (M+H) calc. 316.1913, found 316.1912.

### Example 6

### 7β-Methyl-7,8-dihydromorphine (9)

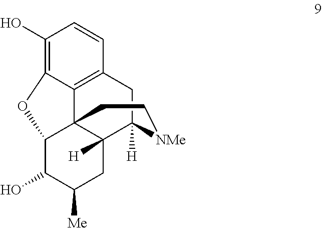

As shown in FIG. 3B, to a stirred solution of 8 (27 mg as HCl salt, 0.077 mmol) in dichloromethane (2mL) at 0° C. under argon was slowly added a solution of BBr₃ in dichloromethane (0.4 ml, 1M). The reaction was monitored by TLC allowing it to gradually warm to 25° C. The reaction was stirred at room temperature for 2 hr and quenched with MeOH (0.5 ml), and saturated aqueous NaHCO₃ solution (2 ml). The mixture was extracted with dichloromethane (4×20 mL), dried (Na₂SO₄), filtered and concentrated in vacuo. The crude product was purified by preparative TLC to give 9 (10 mg, 43%). IR (thin film) 3376, 2930, 1610, 1456 cm⁻¹. ¹H NMR (300 MHz, CDCl₃) δ 6.71 (1H, d, J=8.1 Hz), 6.62 (1H, d, J=8.1 Hz), 4.89 (2H, bs), 4.69 (1H, d, J=4.5 Hz), 3.51 (2H, m), 3.11 (1H, d, J=19.2 Hz), 2.97-2.89 (1H, dd, J's=4.5 and 3.9 Hz), 2.84-2.74 (1H, m), 2.72 (3H, s), 2.53-2.45 (1H, dt, J's=2.7 and 6.6 Hz), 2.07-2.01 (1H, dt, J's=5.1 and 8.1 Hz), 1.83 (1H, d, J=12.8 Hz), 1.36-1.17 (4H, m), 0.90 (3H, d, J=13.5 Hz). ¹³C NMR (75 MHz, CDCl₃) δ 147.2, 140.0, 130.6, 125.3, 120.6, 119.0, 92.0, 73.5, 62.1, 47.6, 43.4, 42.2, 36.5, 35.3, 30.8, 29.5, 22.0, 18.9. HRMS: C₁₈H₂₃NO₃ requires (M+H) calc. 302.1751, found 302.1753.

### Example 7

### Compounds 10′ and 10

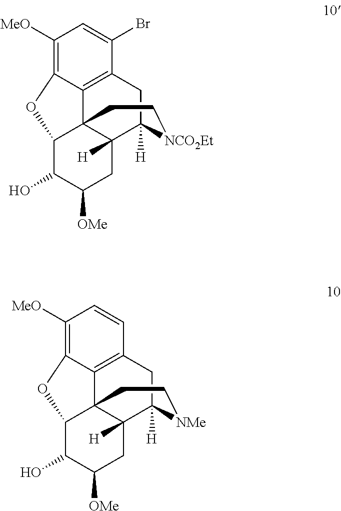

To a stirred solution of 6 (100 mg, 0.23 mmol) in anhydrous MeOH (5 ml) was added p-toluenesulfonic acid (20 mg, 0.11 mmol.) and the solution was heated at gentle reflux until all starting material was consumed as judged by TLC. Upon completion, the reaction solution was concentrated in vacuo and purified by preparative TLC to yield 10′ (88 mg, 68%). IR (thin film) 3442, 2929, 1696, 1683, 1436 cm⁻¹. ¹H NMR (300 MHz, CDCl₃) δ 6.95 (1H, s), 4.75 (0.5H, bs), 4.56 (0.5H, bs), 4.70 (1H, d, J=5.7 Hz), 4.24-4.03 (4H, m), 3.86 (3H, s), 4.51-3.37 (1H, m), 3.33 (3H, d, J=7.2 Hz), 2.83-2.57 (3H, m), 2.40-2.25 (2H, m), 1.87-1.60 (3H, m), 1.35-1.17 (4H, m). ¹³C NMR (75 MHz, CDCl₃) δ 155.7, 146.1, 143.0, 131.2, 125.4, 116.7, 112.9, 89.7, 68.2, 61.8, 60.6, 56.9, 56.7, 51.0, 42.3, 38.0, 36.2, 34.6, 30.3, 23.0, 15.0. HRMS: C₂₁H₂₆BrNO₆ requires (M+Na) calc. 490.0841, found 490.0837.

Compound 10. IR (thin film) 3368, 2928, 1504, 1448, 1276 cm⁻¹. ¹H NMR (300 MHz, CDCl₃) δ 6.71 (1H, d, J=8.4 Hz), 6.62 (1H, d, J=8.4 Hz), 4.85 (1H, m), 4.71 (1H, d, J=5.4 Hz), 3.98 (1H, t, J=5.7 Hz), 3.85 (3H, s), 3.28 (3H, s), 3.27-2.23 (1H, m), 3.14 (1H, dd, J's=2.1 and 3.0 Hz), 3.02 (1H, d, J=18.6 Hz), 2.64-2.51 (2H, m), 2.48-2.38 (1H, m), 2.47 (3H, s), 2.28 (1H, dt, J's=3.6 and 8.7 Hz), 1.97 (1H, dt, J's=4.8 and 7.5 Hz), 1.72-1.57 (2H, m), 1.38-1.29 (1H, m). ¹³C NMR (75 MHz, CDCl₃) δ 146.3, 142.1, 130.3, 126.4, 119.4, 113.7, 89.7, 69.2, 59.9, 56.7, 56.6, 47.1, 43.0, 41.7, 36.5, 35.4, 28.5, 24.3, 20.5. HRMS: C₁₉H₂₅NO₄ requires (M+H) talc. 332.1862, found 332.1829.

### Example 8

### Compounds 11′ and 11

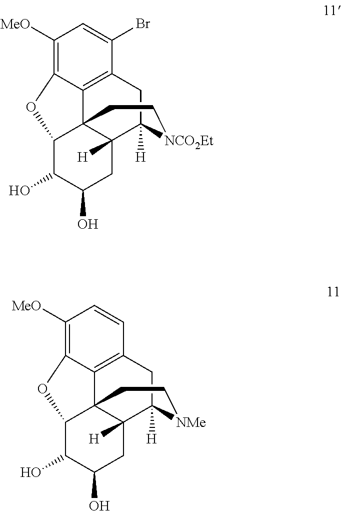

To a stirred solution of 6 (100 mg, 0.23 mmol) in water (2 mL) and THF (2 mL) was added methanesulfonic acid (3 drops) and the solution was heated at gentle reflux until all starting material was consumed as judged by TLC. Upon completion, the solution was concentrated in vacuo and extracted with EtOAc (3×30 mL). The solution was dried (Na₂SO₄), filtered and concentrated in vacuo. Purification of the crude product by preparative TLC gave 11′ (100 mg, 99%).

Compound 11′. IR (thin film) 3436, 2934, 1676, 1487, 1435 cm⁻¹. ¹H NMR (300 MHz, CDCl₃) δ 6.97 (1H, s), 4.80 (1H, d, J=4.8 Hz), 4.78 (0.6H, bs), 4.63 (0.4H, bs), 4.24-4.10 (2H, m), 4.10-3.92 (1H, m), 3.88 (3H, s), 3.78-3.71 (1H, m), 3.46 (1H, dd, J's=7.8 and 7.5 Hz), 2.94-2.70 (2H, m), 2.62 (1H, d, J=19.2 Hz), 2.44-2.39 (1H, m), 2.24 (2H, bs), 1.90-1.70 (2H, m), 1.70-1.60 (1H, dd, J's=6.0 and 6.6 Hz), 1.45-1.41 (1H, m), 1.23 (3H, m). ¹³C NMR (75 MHz, CDCl₃) δ 155.3, 145.4, 143.0, 130.7, 125.7, 116.8, 113.1, 90.3, 71.5, 66.9, 61.7, 56.5, 50.4, 43.4, 37.2, 36.4, 34.6, 30.1, 28.4, 14.6. HRMS: C₂₀H₂₄BrNO₆ requires (M+H) calc. 454.0860, found 454.0867.

Compound 11. IR: 3383, 2928, 1505, 1451, 1277, 1073 cm⁻¹. ¹H NMR (300 MHz, CDCl₃) δ 6.74 (1H, d, J=8.1 Hz), 6.65 (1H, d, J=8.4 Hz), 4.79 (1H, d, J=5.1 Hz), 3.85 (3H, s), 3.80-3.72 (1H, m), 3.54-3.32 (4H, m), 3.23-3.16 (1H, m), 3.01 (1H, d, J=19.2 Hz), 2.71-2.48 (2H, m), 2.44 (3H, s), 2.30 (1H, dt, J's=3.6 and 6.0 Hz), 2.01 (1H, dt, J's=4.8 and 7.8 Hz), 1.72 (1H, d, J=12.6 Hz), 1.61-1.36 (2H, m). ¹³C NMR (75 MHz, CDCl₃) δ 146.1, 142.1, 130.3, 126.9, 119.7, 113.9, 90.5, 71.8, 67.1, 59.9, 56.7, 46.7, 43.0, 42.5, 36.7, 35.6, 29.2, 20.3. HRMS: C₁₈H₂₃NO₄ requires (M+H) calc. 318.1705, found 318.1702.

### Example 9

### Compounds 12′ and 12

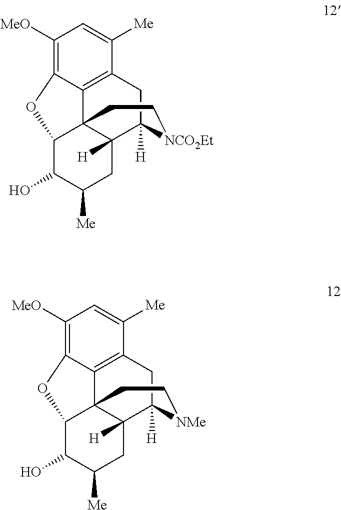

A solution of 7 (100 mg, 0.22 mmol), methyl boronic acid (53 mg, 0.88 mmol), BHT (25 mg, 0.11 mmol), and K₂CO₃ (185 mg, 1.32 mmol) in dioxane (15 mL) and water (5 mL) was heated to 75° C. under argon for 5 min. After addition of Pd(dppf)Cl₂ the mixture was heated at reflux for 15 min. The solution was cooled to room temperature, diluted with water (10 mL) and extracted with EtOAc (3×10 mL), dried (Na₂SO₄) and filtered. The filtrate was concentrated in vacuo to afford 12′ (70 mg, 80%). IR (thin film) 3447, 2932, 1684 cm⁻¹. ¹H NMR (300 MHz, CDCl₃) δ 6.61 (1H, s), 4.71 (0.5H, bs), 4.55 (0.5H, bs), 4.67 (1H, d, J=4.5 Hz), 4.22-4.10 ((2H, m), 4.10-3.90 (1H, m), 3.87 (3H, s), 3.56-3.48 (1H, m), 2.91-2.79 (2H, m), 2.54 (1H, d, J=18.6 Hz), 2.21-2.05 (2H, m), 2.17 (3H, s), 1.85-1.70 (2H, m), 1.45-1.15 (3H, m), 1.27 (3H, t, J=14.1 Hz), 0.91 (3H, d, J=6.6 Hz). ¹³C NMR (75 MHz, CDCl₃) δ 156.6, 144.5, 141.8, 129.8, 128.5, 124.5, 114.9, 90.9, 72.7, 61.7, 56.6, 50.9, 43.4, 37.7, 37.6, 37.3, 30.0, 29.1, 28.2, 18.3, 18.0, 15.0. HRMS: C₂₂H₂₉NO₅ requires (M+H) calc. 388.2124, found 388.2117.

### Compound 12

To a solution of 12′ (75 mg, 0.19 mmol) in THF (5 mL) at room temperature under argon was added a solution of LAH in THF (0.4 mL, 2M). The reaction was stirred and monitored by TLC. Upon completion, Rochelle's salt (saturated solution, 5 mL) was added gradually and the resulting mixture extracted with dichloromethane (4×10 mL). The combined extracts were dried (Na₂SO₄) and filtered. The filtrate was concentrated in vacuo and purified by preparative TLC to give 12 (34 mg, 52%). IR: 3378, 2929, 1502, 1447 cm⁻¹. ¹H NMR (300 MHz, CDCl₃) δ 6.59 (1H, s), 4.71 (1H, d, J=4.8 Hz), 3.87 (3H, s), 3.51 (1H, m), 3.17 (1H, m), 2.83 (1H, d, J=18.6 Hz), 2.62-2.54 (1H, m), 2.46 (3H, s), 2.43-2.24 (3H, m), 2.21 (3H, s), 2.07-1.92 (1H, m), 1.74 (1H, d, J=12 Hz), 1.38-1.16 (4H, m), 0.91 (3H, d, J=6.3 Hz). ¹³C NMR (75 MHz, CDCl₃) δ 144.5, 141.6, 130.7, 128.0, 125.4, 114.7, 91.1, 72.9, 60.0, 56.7, 46.6, 43.3, 42.9, 37.4, 35.7, 29.7, 29.6, 19.1, 18.6, 18.1. HRMS: C₂₀H₂₇NO₃ requires (M+Na) calc. 352.1889, found 352.1884.

### Example 10

### Compounds 13′ and 13

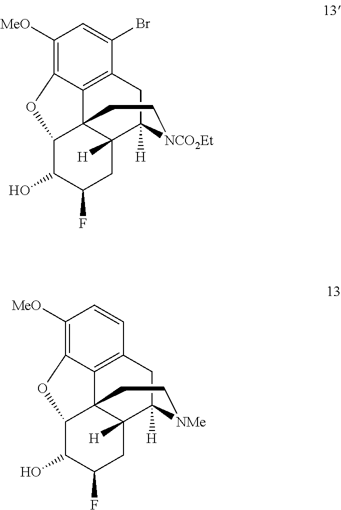

To a solution of 6 (100 mg, 0.22 mmol) in dichloromethane (5 mL), was added a solution of HF.pyridine (2 mL) and the mixture stirred at room temperature under argon. More reagent was added if reaction was found to progress too slowly. Upon completion, as judged by TLC, the reaction was diluted with water (10 mL) and extracted with dichloromethane (3×10 mL). The mixture was dried (Na₂SO₄) and filtered. After concentration in vacuo, the crude product was purified by preparative TLC to give 13′ (70 mg, 65%). IR (thin film) 3447, 2952, 1684, 1488 cm⁻¹. ¹H NMR (300 MHz, CDCl₃) δ 6.96 (1H, s), 4.75 (2H, d, J=5.1 Hz), 4.59 (1H, bs), 4.24-4.10 (3H, m), 4.09-3.90 (1H, m), 3.87 (3H, s), 2.85-2.60 (3H, m), 2.51-2.38 (2H, m), 1.91-1.65 (3H, m), 1.34-1.20 (4H, m). ¹³C NMR (75 MHz, CDCl₃) δ 155.6, 145.9, 143.1, 130.6, 125.3, 116.9, 113.2, 90.9, 89.2, 88.6, 67.7, 67.3, 61.9, 56.7, 50.5, 42.6, 37.9, 34.3, 30.1, 15.3. HRMS: C₂₀H₂₃BrFNO₅ requires (M+H) calc. 458.0978, found 458.0799.

### Compound 13

To a solution of 13′ (60 mg, 0.13 mmol) in THF (2 mL) at room temperature under an argon atmosphere was added a solution of LAH (0.3 mL, 2M) in THF. The reaction was stirred and monitored by TLC. Upon completion, it was slowly quenched with a saturated solution of Rochelle's salt (2 ml), and extracted with dichloromethane (4×5 mL). Purification by preparative TLC gave 13 (19 mg, 45%). IR (thin film) 3367, 2917, 1505, 1450, 1277, 1046 cm⁻¹. ¹H NMR (300 MHz, CDCl₃) δ 6.77 (1H, d, J=8.4 Hz), 6.61 (1H, d, J=8.1 Hz), 4.80 (1H, m), 4.55 (0.5H, m), 4.39 (0.5H, m), 3.88 (3H, s), 3.35-3.18 (1H, m), 3.27 (1H, m), 3.08 (1H, d, J=18.9 Hz), 2.80-2.65 (2H, m), 2.60-2.45 (1H, m), 2.52 (3H, s), 2.43-2.32 (1H, m), 2.14-2.00 (1H, m), 1.90-1.65 (3H, m), 1.58-1.38 (1H, m). ¹³C NMR (75 MHz, CDCl₃) δ 146.0, 142.4, 129.5, 125.9, 120.0, 114.1, 90.8, 89.2, 88.6, 68.8, 68.4, 59.7, 56.6, 47.0, 42.8, 41.8, 36.0, 20.5. HRMS: C₁₈H₂₂FNO₃ requires (M+H) calc. 320.1662, found 320.1659.

### Example 11

### Compounds 14′ and 14

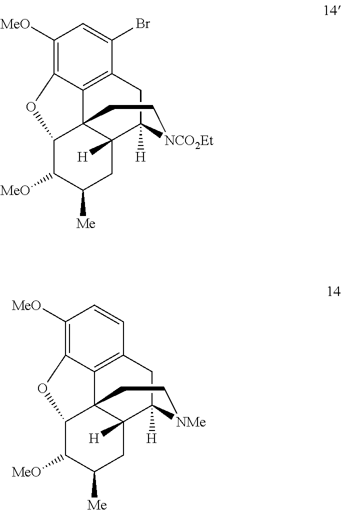

To a stirred solution of 7 (60 mg, 0.14 mmol) in THF (5 mL) was added imidazole (10 mg, 0.14 mmol), along with KH (56 mg, 1.4 mmol). After 5 min of stirring MeI (70 μL, 1.4 mmol) was added and the reaction stirred at room temperature to completion as judged by TLC. Upon completion, the reaction was quenched with MeOH (1 mL) and water (10 mL). The mixture was then extracted with EtOAc (3×10 mL), dried (Na₂SO₄), and filtered. The filtrate was concentrated in vacuo and purified by preparative TLC to give 14′ (37 mg, 53%). IR (thin film) 2934, 1694, 1487, 1435 cm⁻¹. ¹H NMR (300 MHz, CDCl₃) δ 6.94 (1H, s), 4.82 (1H, d, J=4.5 Hz), 4.70 (0.6H, bs), 4.55 (0.4H, bs), 4.25-4.10 (2H, m), 4.10-3.88 (1H, m), 3.86 (3H, s), 3.41 (3H, s), 3.20-3.10 (1H, m), 2.90-2.75 (2H, m), 2.59 (1H, d, J=18.9 Hz), 2.20-2.10 (1H, m), 1.82-1.70 (2H, m), 1.71-1.60 (1H, m), 1.35-1.10 (5H, m), 0.90 (3H, d, J=6 Hz). ¹³C NMR (75 MHz, CDCl₃) δ 155.6, 146.9, 143.2, 131.0, 116.8, 112.1, 108.0, 88.5, 81.5, 61.8, 61.7, 58.5, 56.7, 51.0, 43.5, 37.7, 35.3, 30.7, 29.0, 28.2, 17.8, 14.9. HRMS: C₂₂H₂₈BrNO₅ requires (M+H) talc. 466.1229, found 466.1224.

### Compound 14

A solution of 14′ (37 mg, 0.08 mmol) in THF (2 mL) was treated with LAH in THF (0.25 mL, 2M) at room temperature under argon. Upon completion, as judged by TLC, the solution was quenched with a saturated solution of Rochelle's salt (5 mL), and extracted with dichloromethane (4×10 mL). The combined extracts were dried (Na₂SO₄), filtered and concentrated in vacuo. The crude sample was purified by preparative TLC to give 14 (11 mg, 42%). IR (thin film) 3420, 2928, 1506, 1456 cm⁻¹. ¹H NMR (300 MHz, CDCl₃) δ 6.78 (1H, d, J=9.5 Hz), 6.65 (1H, d, J=9.5 Hz), 4.87 (1H, d, J=4.8 Hz), 3.87 (3H, s), 3.45 (3H, s), 3.38-3.32 (1H, m), 3.18 (1H, q, J=4.5 Hz), 3.03 (1H, d, J=18.6 Hz), 2.90-2.78 (1H, m), 2.77-2.62 (2H, m), 2.61 (3H, s), 2.57-2.47 (1H, m), 2.28-2.15 (1H, m), 1.82 (1H, dd, J's=2.4 and 10.5 Hz), 1.63-1.53 (1H, m), 1.34-1.17 (2H, m), 0.91 (3H, d, J=6.9 Hz). ¹³C NMR (75 MHz, CDCl₃) δ 146.0, 142.6, 134.0, 132.0, 119.0, 115.8, 87.7, 81.5, 60.8, 58.3, 56.6, 47.1, 42.6, 42.1, 36.0, 34.0, 28.7, 28.4, 21.0, 18.1. HRMS: C₂₀H₂₇NO₃ requires (M+H) calc. 330.2069, found 330.2065.

### REFERENCES

1. Magnus, P. and Ghavimi-Alagha, B. “New Chemical Transformation of (−)-Codeine to Afford New Derivatives of Codeine and Morphine Thereof,” U.S. Provisional Patent Application 61/656,817, filed Jun. 7, 2012. (published N/A).
2. Abdel-Monem, M. M. and Portoghese, P. S. (1972) “N-Demethylation of morphine and structurally related compounds with chloroformate esters,” J. Med. Chem. 15(2), 208-210.
3. Myers, A. G and Zheng, B. (1996) “An efficient method for the reductive transposition of allylic alcohols,” Tetrahedron Lett. 37(28), 4841-4844.
4. Magnus, P. et al. (2009) “Concise Syntheses of (−)-Galanthamine and (±)-Codeine via Intramolecular Alkylation of a Phenol Derivative,” J. Am. Chem. Soc. 131(44), 16045-16047.
5. Stahl, P. H. and Wermuth, C. G, (Eds.) (2002) Handbook of Pharmaceutical Salts: Properties Selection and Use, Verlag Helvetica Chimica Acta/Wiley-VCH, Zurich.
6. Elger, G. A. et al. “Controlled release pharmaceutical composition,” U.S. Pat. No. 4,828,836, application Ser. No. 07/052,580, filed May 19, 1987. (issued May 9, 1989).
7. Van Lengerich, B. H. “Embedding and encapsulation of controlled release particles,” U.S. Pat. No. 6,190,591, application Ser. No. 09/269,763, filed May 17, 1999. (issued Feb. 20, 2001).
8. Magnus, P. D. et al. “Efficient Synthesis Of Morphine And Codeine,” United States Patent Application Publication Number 20100292475, application Ser. No. 12/778,466, filed May 12, 2010. (published Dec. 18, 2010).
9. Zezula, J. et al. (2007) “Design for Morphine Alkaloids by Intramolecular Heck Strategy: Chemoenzymatic Synthesis of 10-Hydroxy-14-epi-dihydrocodeinone via C-D-B Ring Construction,” Synlett 18, 2863-2867.
10. Omori, A. T. et al. (2007) “Chemoenzymatic Total Synthesis of (+)-Codeine by Sequential Intramolecular Heck Cyclizations via C-B-D Ring Construction,” Synlett 18, 2859-2862.
11. Uchida, K. et al. (2006) “Total Synthesis of (±)-Morphine,” Org. Lett. 8(23), 5311-5313.
12. Trost, B. M. et al. (2005) “Divergent Enantioselective Synthesis of (−)-Galanthamine and (−)-Morphine,” J. Am. Chem. Soc. 127(42), 14785-14803.
13. Rice, K. C. (1980) “Synthetic opium alkaloids and derivatives. A short total synthesis of (.+−.)-dihydrothebainone, (.+−.)-dihydrocodeinone, and (.+−.)-nordihydrocodeinone as an approach to a practical synthesis of morphine, codeine, and congeners,” J. Org. Chem. 45(15), 3135-3137.
14. Grosswald, R. R. et al. “Method for the manufacture of pharmaceutical cellulose capsules,” U.S. Pat. No. 5,698,155, application Ser. No. 08/377,669, filed Jan. 24, 1995. (issued Dec. 16, 1997).
15. PDR Staff (2008) Physician's Desk Reference, 62nd Edition.
16. Perrin, D. D. and Armarego, W. L. F. (1988) Purification of Laboratory Chemicals, 3rd ed., Pregamon Press, Oxford.

1. A method of preparing a carbamate derivative, comprising:
a) providing (−)-codeine phosphate;
b) treating said codeine phosphate derivative under conditions so as to create a carbamate derivative;
c) treating said carbamate derivative with reducing agent, so as to create a 6,7-alkene derivative.

2. The method of claim 1, wherein said step b) comprises treating said (−)-codeine phosphate with ClCO₂Et, K₂CO₃, and chloroform in reflux.

3. The method of claim 1, wherein said (−)-codeine phosphate has the structure:

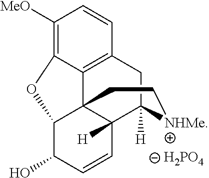

4. The method of claim 1, wherein said stereospecific enantiomer carbamate derivative has the structure:

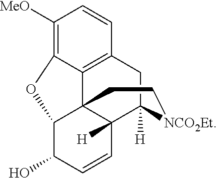

5. (canceled)

6. The method of claim 1, wherein said 6,7-alkene derivative has the structure:

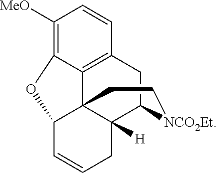

7. The method of claim 1, further comprising d) treating said 6,7-alkene derivative in dioxane and water at −10° C. and 1,3-dibromo-5,5-dimethylhydantoin, so as to create a bromohydrin.

8. The method of claim 7, wherein said bromohydrin has the structure:

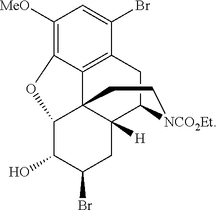

9. The method of claim 7, further comprising e) treating said halohydrin with KOH, so as to create a 6,7-epoxide derivative.

10. The method of claim 9, wherein said 6,7-epoxide derivative has the structure:

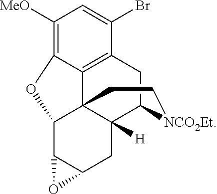

11. The method of claim 9, further comprising f) treating said 6,7-epoxide derivative under such conditions to create a ring opened derivative with the structure:

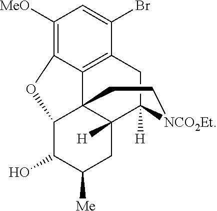

12. The method of claim 11, wherein said conditions comprise in dichloromethane and water with Me₃Al.

13. The method of claim 11, further comprising g) treating said ring opened derivative under reducing conditions, so as to create a tertiary amine derivative with the structure:

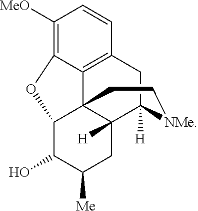

14. The method of claim 13, wherein said conditions comprise LiAlH₄ in THF at 0° C.

15. The method of claim 9, further comprising f) treating said 6,7-epoxide derivative under such conditions to create a ring opened derivative with the structure:

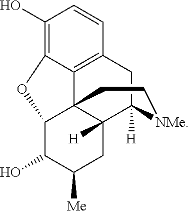

16. The method of claim 15, wherein said conditions comprise BBr₃ in CH₂Cl₂ under temperatures between 0 to 25° C.

17. The method of claim 9, further comprising f) treating said 6,7-epoxide derivative in anhydrous MeOH with p-toluenesulfonic acid added and refluxed to create a ring opened derivative with the structure:

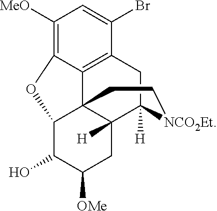

18. The method of claim 17, further comprising g) treating said ring opened derivative under such reducing conditions to create a ring opened derivative with the structure:

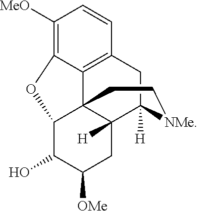

19. The method of claim 18, wherein said reducing conditions comprise lithium aluminum hydride in THF at room temperature under argon.

20. The method of claim 9, further comprising f) treating said 6,7-epoxide derivative in water and THF with methanesulfonic acid added and refluxed to create a ring opened derivative with the structure:

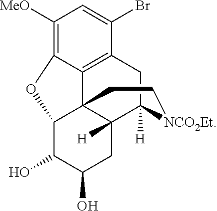

21. The method of claim 20, further comprising g) treating said ring opened derivative under such reducing conditions to create a ring opened derivative with the structure:

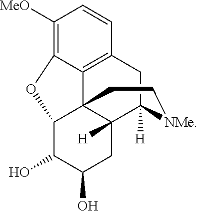

22. The method of claim 21, wherein said reducing conditions comprise lithium aluminum hydride in THF at room temperature under argon.

23. The method of claim 9, further comprising f) treating said 6,7-epoxide derivative in dichloromethane with HF pyridine added and the mixture stirred at room temperature under argon to create a ring opened derivative with the structure:

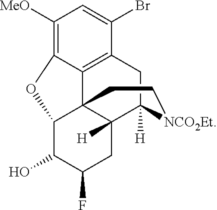

24. The method of claim 23, further comprising g) treating said ring opened derivative under such reducing conditions to create a ring opened derivative with the structure:

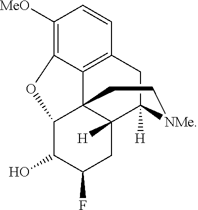

25. The method of claim 9, further comprising f) treating said 6,7-epoxide derivative in THF with imidazole and KH added with subsequent addition of methyl iodide and the mixture stirred at room temperature under argon to create a ring opened derivative with the structure:

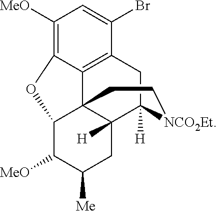

26. The method of claim 25, further comprising g) treating said ring opened derivative under such reducing conditions to create a ring-opened derivative with the structure:

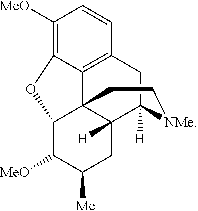

27. The method of claim 26, wherein said reducing conditions comprise lithium aluminum hydride in THF at room temperature under argon.

28-40. (canceled)
